(19) United States
(12) Patent Application Publication
(10) Pub. No.: US 2022/0118121 A1
(43) Pub. Date: Apr. 21, 2022

(54) PROSTATE SPECIFIC MEMBRANE ANTIGEN (PSMA) LIGANDS WITH IMPROVED TISSUE SPECIFICITY

(71) Applicants: Ruprecht-Karls-Universität Heidelberg, Heidelberg (DE); Deutsches Krebsforschungszentrum, Heidelberg (DE)

(72) Inventors: Matthias Eder, Simonswald (DE); Ann-Christin Baranski, Freiburg (DE); Michael Eisenhut, Heidelberg (DE); Uwe Haberkorn, Schwetzingen (DE)

(21) Appl. No.: 17/431,396

(22) PCT Filed: Feb. 14, 2020
(86) PCT No.: PCT/EP2020/053925

§ 371 Date: Aug. 16, 2021

(30) Foreign Application Priority Data
Feb. 14, 2019 (EP) 19157214.8

(51) Int. Cl. A61K 51/08; A61K 51/04; A61P 35/00

(52) U.S. Cl. CPC A61K 51/088; A61P 35/00; A61K 51/0497

(57) ABSTRACT

The present invention relates to a compound of formula (1), and to a complex comprising said compound and a radionuclide, and to the respective pharmaceutical composition, the compound having the following structure

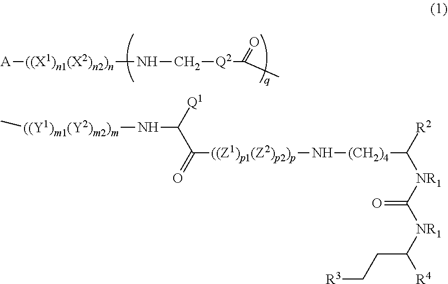

or a pharmaceutically acceptable salt or solvate thereof, wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂, Q¹ is selected from the group consisting of alkylaryl, arylalkyl, aryl, alkylheteroaryl, heteroarylalkyl and heteroaryl, Q² is selected from the group consisting of aryl, alkylaryl, arylalkyl, cycloalkyl, heterocycloalkyl, heteroaryl, heteroarylalkyl and alkylheteroaryl, A is a chelator residue derived from a chelator selected from the group consisting of 1,4,7,10-tetraazacyclododecane-N,N′,N″,N′″-tetraacetic acid (=DOTA), N,N″-bis[2-hydroxy-5-(carboxyethyl)benzyl]ethylenediamine-N,N″-diacetic acid, 1,4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7, 10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid](NOPO), 3,6,9, 15-tetraazabicyclo[9.3.1.]pentadeca-1(15),11,13-triene-3,6,9-triacetic acid (=PCTA), N′-{5-[Acetyl(hydroxy)amino]pentyl}-N-[5-({4-[(5-aminopentyl)(hydroxy)amino]-4-oxobutanoyl}amino)pentyl]-N-hydroxysuccinamide (DFO), Diethylenetriaminepentaacetic acid (DTPA), Trans-cyclohexyl-diethylenetriaminepentaacetic acid (CHX-DTPA), 1-oxa-4,7, 10-triazacyclododecane-4,7, 10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl-DTPA (MX-DTPA), X¹, X², Y¹, Y², Z¹ and Z², are independently of each other, charged amino acids, q is an integer of from 0-3, n, m and p, are independently of each other an integer of from 0 to 9, n1, n2, m1, m2, p1, p2, are independently of ach other, an integer of from 0 to 3, and wherein n1+n2>0, m1+m2>0 and p1+p2>0, and wherein n+m+p>0. Further, the present invention relates to the compound, the complex, and the pharmaceutical composition for use in treating, ameliorating or preventing PSMA-expressing cancers, in particular prostate cancer, and/or metastases thereof.

### FIELD OF THE INVENTION

The present invention generally relates to the field of radiopharmaceuticals and their use in nuclear medicine as tracers, imaging agents and for the treatment of various disease states of PSMA-expressing cancers, especially prostate cancer, and metastases thereof.

### RELATED ART

Prostate cancer (PCa) is the leading cancer in the US and European population. At least 1-2 million men in the western hemisphere suffer from prostate cancer and it is estimated that the disease will strike one in six men between the ages of 55 and 85. There are more than 300,000 new cases of prostate cancer diagnosed each year in USA. The mortality from the disease is second only to lung cancer. Currently, imaging methods with high resolution of the anatomy, such as computed tomography (CT), magnetic resonance (MR) imaging and ultrasound, predominate for clinical imaging of prostate cancer. An estimated annual $2 billion is currently spent worldwide on surgical, radiation, drug therapy and minimally invasive treatments. However, there is presently no effective therapy for relapsing, metastatic, androgen-independent prostate cancer.

A variety of experimental low molecular weight PCa imaging agents are currently being pursued clinically, including radiolabeled choline analogs [¹⁸F]fluorodihydrotestosterone ([¹⁸F]FDHT), anti-1-amino-3-[¹⁸F]fluorocyclobutyl-1-carboxylic acid (anti[¹⁸F]F-FACBC, [¹¹C]acetate and 1-(2-deoxy-2-[¹⁸F]flouro-L-arabinofuranosyl)-5-methyluracil (—[¹⁸F]FMAU)(Scher, B.; et al. Eur J Nucl Med Mol Imaging 2007, 34, 45-53; Rinnab, L; et al. BJU Int 2007, 100, 786,793; Reske, S. N.; et al. J Nucl Med 2006, 47, 1249-1254; Zophel, K.; Kotzerke, J. Eur J Nucl Med Mol Imaging 2004, 31, 756-759; Vees, H.; et al. BJU Int 2007, 99, 1415-1420; Larson, S. M.; et al. J Nucl Med 2004, 45, 366-373; Schuster, D. M.; et al. J Nucl Med 2007, 48, 56-63; Tehrani, O. S.; et al. J Nucl Med 2007, 48, 1436-1441). Each operates by a different mechanism and has certain advantages, e.g., low urinary excretion for [¹¹C]choline, and disadvantages, such as the short physical half-life of positron-emitting radionuclides.

It is well known that tumors may express unique proteins associated with their malignant phenotype or may over-express normal constituent proteins in greater number than normal cells. The expression of distinct proteins on the surface of tumor cells offers the opportunity to diagnose and characterize disease by probing the phenotypic identity and biochemical composition and activity of the tumor. Radioactive molecules that selectively bind to specific tumor cell surface proteins provide an attractive route for imaging and treating tumors under non-invasive conditions. A promising new series of low molecular weight imaging agents targets the prostate-specific membrane antigen (PSMA) (Mease R. C. et al. Clin Cancer Res. 2008, 14, 3036-3043; Foss, C. A.; et al. Clin Cancer Res 2005, 11, 4022-4028; Pomper, M. G.; et al. Mol Imaging 2002, 1, 96-101; Zhou, J.; et al. Nat Rev Drug Discov 2005, 4, 015-1026; WO 2013/022797).

PSMA is a trans-membrane, 750 amino acid type II glycoprotein that has abundant and restricted expression on the surface of PCa, particularly in androgen-independent, advanced and metastatic disease (Schulke, N.; et al. Proc Natl Acad Sci USA 2003, 100, 12590-12595). The latter is important since almost all PCa become androgen independent over the time. PSMA possesses the criteria of a promising target for therapy (Schulke, N.; et al. Proc. Natl. Acad. Sci. USA 2003, 100, 12590-12595). The PSMA gene is located on the short arm of chromosome 11 and functions both as a folate hydrolase and neuropeptidase. It has neuropeptidase function that is equivalent to glutamate carboxypeptidase II (GCPII), which is referred to as the “brain PSMA”, and may modulate glutamatergic transmission by cleaving/V-acetylaspartylglutamate (NAAG) to N-acetylaspartate (NAA) and glutamate (Nan, F.; et al. J Med Chem 2000, 43, 772-774). There are up to 10⁶ PSMA molecules per cancer cell, further suggesting it as an ideal target for imaging and therapy with radionuclide-based techniques (Tasch, J.; et al. Crit Rev Immunol 2001, 21, 249-261).

The radio-immunoconjugate of the anti-PSMA monoclonal antibody (mAb) 7E11, known as the PROSTASCINT® scan, is currently being used to diagnose prostate cancer metastasis and recurrence. However, this agent tends to produce images that are challenging to interpret (Lange, P. H. PROSTASCINT scan for staging prostate cancer. Urology 2001, 57, 402-406; Haseman, M. K.; et al. Cancer Biother Radiopharm 2000, 15, 131-140; Rosenthal, S. A.; et al. Tech Urol 2001, 7, 27-37). More recently, monoclonal antibodies have been developed that bind to the extracellular domain of PSMA and have been radiolabeled and shown to accumulate in PSMA-positive prostate tumor models in animals. However, diagnosis and tumor detection using monoclonal antibodies has been limited by the low permeability of the monoclonal antibody in solid tumors.

The selective targeting of cancer cells with radiopharmaceuticals, either for imaging or therapeutic purposes is challenging. A variety of radionuclides are known to be useful for radio-imaging or cancer radiotherapy, including ¹¹¹In, ⁹⁰Y, ⁶⁸Ga, ¹⁷⁷Lu, ^{99m}Tc, ¹²³I and ¹³¹I. Recently it has been shown that some compounds containing a glutamate-urea-glutamate (GUG) or a glutamate-urea-lysine (GUL) recognition element linked to a radionuclide-ligand conjugate exhibit high affinity for PSMA.

In WO 2015/055318 new imaging agents with improved tumor targeting properties and pharmacokinetics were described. These compounds comprise a motif specifically binding to cell membranes of cancerous cells, wherein said motif comprises a prostate-specific membrane antigen (PSMA), that is the above mentioned glutamate-urea-lysine motif. The preferred molecules described in WO 2015/055318 further comprise a linker which binds via an amide bond to a carboxylic acid group of DOTA as chelator. Some of these compounds have been shown to be promising agents for the specific targeting of prostate tumors. The compounds were labeled with ¹⁷⁷Lu (for therapy purposes) or ⁶⁸Ga (for diagnostic purposes) and allow for visualization and targeting of prostate cancer for radiotherapy purposes.

However, in therapeutic applications of radioactively labeled PSMA inhibitors, organs with physiological PSMA expression turned out to be dose limiting and thus minimize the therapeutic success. In particular, the high renal and salivary gland uptake of the radioactively labeled PSMA inhibitor substances is noticeable, which, in the case of a therapeutic application, gives rise to considerable side effects. Attempts to improve the kidney uptake of PSMA inhibitors has led to the development of PSMA-617 [Benesova, M., et al. (2016) J Med Chem 59, 1761-75], a compound which is already used clinically with 177Lu or 225Ac for endoradiotherapy of prostate cancer. However, a reduction in salivary and lacrimal gland uptake has not yet been achieved and is still described as critical and dose-limiting in early clinical work. In a first-in-man study with 225Ac-PSMA-617, two patients with extremely advanced and end-stage disease showed complete remission. In both patients the PSA value fell below the detectability limit. Accompanying diagnostic recordings with 68Ga-PSMA-11 confirmed a complete response.

As already mentioned above, the strong accumulation of PSMA ligands in the salivary and lacrimal glands, which is described in numerous papers leads to considerable side effects. The salivary and lacrimal glands are severely and partially irreversibly damaged, in particular during alpha therapy with 225Ac. The resulting xerostomia for example represents a dose-limiting side effect.

Thus, there is still the need for improved PSMA ligands which provide advantageous options for the detection, treatment and management of PSMA-expressing cancers, in particular prostate cancer, and which preferably show less side effects on the salivary glands and/or lacrimal glands, in particular which show a reduced salivary gland and/or lacrimal gland uptake thereby reducing the respective side effects.

### SUMMARY OF THE INVENTION

The solution of said object is achieved by providing the embodiments characterized in the claims. The inventors found new compounds which are useful and advantageous radiopharmaceuticals and which can be used in nuclear medicine as tracers, imaging agents and for the treatment of various disease states of PSMA-expressing cancers, in particular prostate cancer. These compounds are described in more detail below:

In particular, the present invention relates to a compound of formula (1)

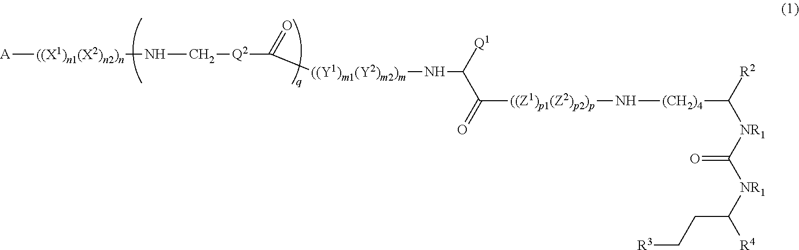

or a pharmaceutically acceptable salt or solvate thereof, wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂, Q¹ is selected from the group consisting of alkylaryl, arylalkyl, aryl, alkylheteroaryl, heteroarylalkyl and heteroaryl, Q² is selected from the group consisting of aryl, alkylaryl, arylalkyl, cycloalkyl, heterocycloalkyl, heteroaryl, heteroarylalkyl and alkylheteroaryl, A is a chelator residue derived from a chelator selected from the group consisting of 1,4,7,10-tetraazacyclododecane-N,N′,N″,N′″-tetraacetic acid (=DOTA), N,N″-bis[2-hydroxy-5-(carboxyethyl)benzyl]ethylenediamine-N,N″-diacetic acid, 1,4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7, 10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid](NOPO), 3,6,9, 15-tetraazabicyclo[9.3.1.]pentadeca-1(15),11,13-triene-3,6,9-triacetic acid (=PCTA), N′-{5-[Acetyl(hydroxy)amino]pentyl}-N-[5-({4-[(5-aminopentyl)(hydroxy)amino]-4-oxobutanoyl}amino)pentyl]-N-hydroxysuccinamide (DFO), Diethylenetriaminepentaacetic acid (DTPA), Trans-cyclohexyl-diethylenetriaminepentaacetic acid (CHX-DTPA), 1-oxa-4,7, 10-triazacyclododecane-4,7, 10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl-DTPA (MX-DTPA), X¹, X², Y¹, Y², Z¹ and Z², are independently of each other, charged amino acids, q is an integer of from 0-3, n, m and p, are independently of each other an integer of from 0 to 9, n1, n2, m1, m2, p1, p2, are independently of ach other, an integer of from 0 to 3, and wherein n1+n2>0, m1+m2>0 and p1+p2>0, and wherein n+m+p>0.

Further, the present invention relates to a complex comprising

(a) a radionuclide, and
(b) a compound, as described above or below, or a pharmaceutically acceptable salt or solvate thereof.

Further, the present invention relates to a pharmaceutical composition comprising a compound, as described above or below, or a pharmaceutically acceptable salt or solvate thereof, as described above or below, or a complex, as described above or below.

Further, the present invention relates to a compound, as described above or below, or a pharmaceutically acceptable salt or solvate thereof, or a complex, as described above or below, or a pharmaceutical composition as described above or below, for use in treating or preventing PSMA-expressing cancers, in particular prostate cancer, and/or metastases thereof.

X¹, X², Y¹, Y², Z¹ and Z²

As described above, X¹, X², Y¹, Y², Z¹ and Z², are, independently of each other, charged amino acids. The term “charged amino acids” as used herein refers to an amino acid that comprises a side chain that is negatively charged (i.e., de-protonated) or positively charged (i.e., protonated) in aqueous solution at physiological pH. It is to be understood that the term includes naturally-occurring and non-naturally-occurring charged amino acids, including all stereoisomers, such as enantiomers and diastereomers of these amino acids. Most preferably, the amino acids are alpha amino acids. With respect to the chirality, L-amino acids are preferred.

The term “negatively charged amino acid” includes, but is not limited to, aspartic acid, glutamic acid, cysteic acid, homocysteic acid, and homoglutamic acid, homoglutamic acid, a sulfonic acid derivative of Cys, cysteic acid, homocysteic acid, aspartic acid (D) and glutamic acid (E). More preferably, the negatively charged amino acid is aspartic acid or glutamic acid (E).

The term “sulfonic acid derivative of Cys” preferably refers to an amino acid having the structure HO₂C—CH(NH₂)—CH—S(OH)(═O)₂, CAS NO:498-40-8.

The term “positively charged amino acid” includes, but is not limited to, arginine, lysine, histidine homoarginine, 3- and 4-substituted arginine analogs, N(delta)-methyl-arginine (deltaMA), canavanine, substituted analogs of canavanine, α-Amino-β-guanidinopropionic acid, γ-guanidinobutyric acid, citrulline, 3-guanidinopropionic acid, 4-{[amino(imino)methyl]amino}butanoic acid, 6-{[amino(imino)methyl]amino}hexanoic acid, 2-Amino-3-guanidinopropionic acid, Arginine hydroxamate, Agmatine (CAS #: 2482-00-0), and NG-Methyl-arginine. Most preferred positively charged amino acids are lysine (K), histidine (H) and arginine (R).

Integers n, m and p, n1, n2, m1, m2, p1, p2

As described above, n, m and p are, independently of each other, an integer of from 0 to 9, with the proviso that n+m+p>0.

Further, n1, n2, m1, m2, p1 and p2 are, independently of ach other, an integer of from 0 to 3, wherein n1+n2>0, m1+m2>0 and p1+p2>0.

Thus, n1+n2 is at least 1, m1+m2 is at least 1 and p1+p2 is at least 1. Further, n+m+p is at least 1.

Thus, the compound of formula 1 comprises at least one charged amino acid. In case, (n1+n2)n+(m1+m2)m+(p1+p2)p=1, the compound comprises exactly 1 charged amino acid, that is either X¹, X², Y¹, Y², Z¹ or Z².

Preferably, n1, n2, m1, m2, p1, p2, are independently of each other, 0 or 1.

In particular, the compound comprises at least 2 charged amino acids. Thus, (n1+n2)n+(m1+m2)m+(p1+p2)p is preferably at least 2, with n1, n2, m1, m2, p1, p2, more preferably being, independently of each other, 0 or 1. More preferably, (n1+n2)n+(m1+m2)m+(p1+p2)p is an integer of from 2 to 20, preferably of from 2 to 10, more preferably of from 4 to 8, more preferably 6, with most preferably n1, n2, m1, m2, p1 and p2 being, independently of each other, 0 or 1.

In case, the compound comprises more than 1 amino acid, these amino acids are preferably directly linked with each other via amide bonds, thus forming a peptidic backbone. Thus, preferably, (n1+n2)n>1 and (m1+m2)m+(p1+p2)p=0, or (m1+m2)m>1 and (n1+n2)n+(p1+p2)p=0, or (p1+p2)p>1 and (m1+m2)m+(n1+n2)n=0.

### Preferred Embodiment 1a

According to a first preferred embodiment, (n1+n2)n>1, wherein n1 is preferably 0 or 1 and n2 is preferably 0 or 1.

More preferably, (n1+n2)n is of from 2 to 10, more preferably of from 4 to 8, more preferably 6, with n1 being preferably 0 or 1 and n2 being preferably 0 or 1. It is to be understood that this includes e.g. combination of X¹ and X², such as e.g. ((X¹)₁(X²)₀)₆, ((X¹)₀(X²)₁)₆ as well as, e.g. combinations comprising both amino acids, such as ((X¹)₁(X²)₁)₃.

According to this embodiment, n1 and n2 are preferably both 1 and n is of from 2 to 10, more preferably of from 2 to 5, more preferably 3. According to an alternatively preferred embodiment, n1 and n2 are preferably both 1 and n is preferably 4.

Preferably, at least one of X¹ or X² according to embodiment (1a) is a negatively charged, and at least one of X¹ and X² is a positively charged amino acid.

More preferably, at least one of X¹ and X² is histidine (H) and at least one of X¹ and X² is glutamic acid (E). Even more preferably, the building block ((X¹)ₙ₁(X²)ₙ₂)ₙ, has the structure (HE)ₙ or (EH)ₙ, preferably (EH)ₙ.

More preferably, according to this first embodiment, (m1+m2)m+(p1+p2)p=0. Thus, according to this preferred embodiment, the compound has preferably the structure (1a)

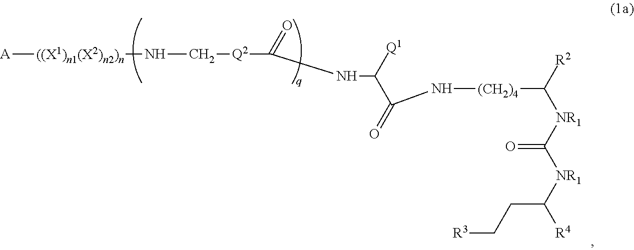

with the building block ((X¹)ₙ₁(X²)ₙ₂)ₙ, more preferably having the structure (HE)ₙ or (EH)ₙ, more preferably (EH)ₙ and with n being most preferably of from 2 to 10, more preferably of from 2 to 5, more preferably 3 or 4. Thus, according to one preferred embodiment, the building block ((X₁)ₙ₁(X²)ₙ₂)ₙ, has the structure (HE)₃ or (EH)₃. According to a further preferred embodiment, the building block ((X¹)ₙ₁(X²)ₙ₂)ₙ, has the structure (HE)₄ or (EH)₄, preferably (HE)₄.

Thus, the following structure (1a_1) is particularly preferred:

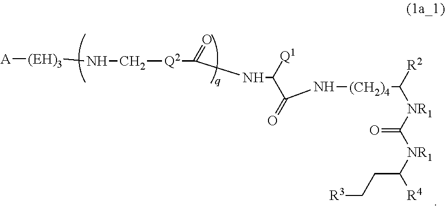

Further, the following structure (1a_2) is preferred:

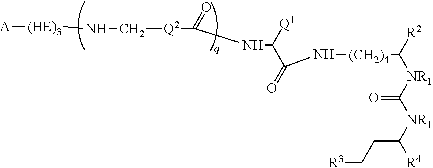

Further, the following structure (1a_3) is particularly preferred:

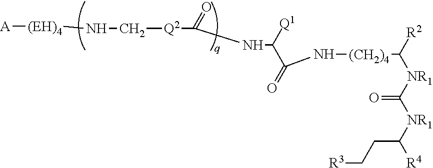

According to a preferred embodiment, the compound is selected from the group consisting of compounds (1a-1), (1a-2) and (1a-3), more preferably (1a-1) or (1a-3).

As outlined above, the amino acids E and H have preferably L-configuration.

### Preferred Embodiment 1aa

According to a further preferred embodiment, (n1+n2)n>1, wherein n1 is preferably 0 or 1 and n2 is preferably 0 or 1.

More preferably, (n1+n2)n is of from 2 to 10, more preferably of from 4 to 8, more preferably 6, with n1 being preferably 0 or 1 and n2 being preferably 0 or 1. It is to be understood that this includes e.g. combination of X¹ and X², such as e.g. ((X¹)₁(X²)₀)₆, ((X¹)₀(X²)₁)₆ as well as, e.g. combinations comprising both amino acids, such as ((X¹)₁(X²)₁)₃.

According to this embodiment, n1 is preferably 1 and n2 is preferably 0, and n is preferably 3 or 4, preferably 3.

X¹ is preferably a negatively charged or a positively charged amino acid, more preferably, X¹ is histidine (H) or glutamic acid (E), more preferably histidine.

More preferably, according to this embodiment (1aa), (m1+m2)m+(p1+p2)p=0. Thus, according to this preferred embodiment, the compound has preferably the structure (1aa)

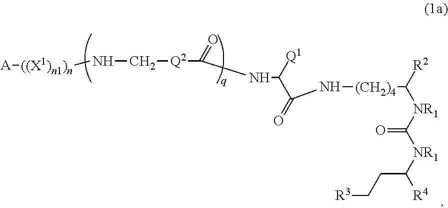

with the building block ((X¹)ₙ₁))ₙ being (H)ₙ or (E)ₙ, more preferably (H)₃ or (E)₃ or (H)₄ or (E)₄, in particular (H)₃ or (E)₃.

Thus, the following structure (1aa_1) is particularly preferred:

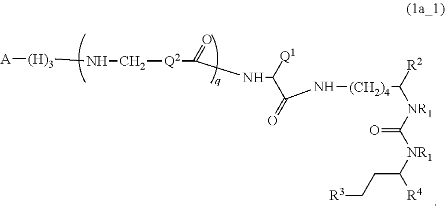

Further, the following structure (1aa_2) is particularly preferred:

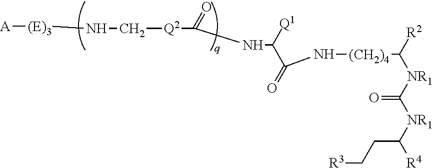

Thus, according to a particularly preferred embodiment, the compound has structure selected from the group consisting of structures (1a_1), (1a_2), (1a_3), (1aa_2) and (1aa_1), more preferably a structure selected from the group consisting of structures (1a_1), (1a_3), (1aa_2) and (1aa_1), more preferably the structure is (1a_1) or (1a_3).

As outlined above, the amino acids E and H have preferably L-configuration.

### Preferred Embodiment 1b

According to a second preferred embodiment, (m1+m2)n>1, wherein m1 is preferably 0 or 1 and m2 is preferably 0 or 1.

More preferably, (m1+m2)m is of from 2 to 10, more preferably of from 4 to 8, more preferably 6, with m1 being preferably 0 or 1 and m2 being preferably 0 or 1. It is to be understood that this includes e.g. combination of Y¹ and Y², such as e.g. ((Y₁)₁(Y²)₀)₆, ((Y¹)₀(Y²)₁)₆ as well as ((Y¹)₁(Y²)₁)₃.

According to embodiment (1b), m1 and m2 are preferably both 1 and m is of from 2 to 10, more preferably of from 2 to 5, more preferably 3.

Preferably, at least one of Y¹ or Y² in this embodiment is a negatively charged, and at least one of Y1 and Y2 is a positively charged amino acid.

More preferably, at least one of Y¹ and Y² is histidine (H) and at least one of Y¹ and Y² is glutamic acid (E). Even more preferably, the building block ((Y¹)ₘ₁(Y²)ₘ₂)ₘ, has the structure (HE)ₘ or (EH)ₘ, preferably (EH)ₘ.

More preferably, according to embodiment (1b), (n1+n2)ₙ+(p1+p2)p=0. Thus, according to this preferred embodiment, the compound has preferably the structure (1b)

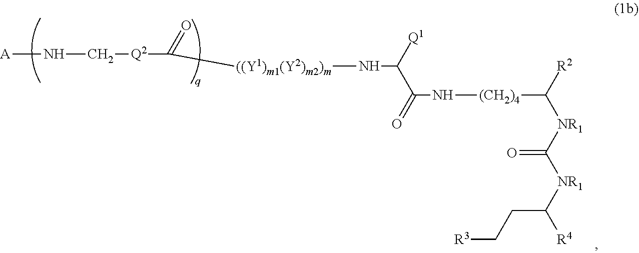

with the building block ((Y¹)ₘ₁(Y²)ₘ₂)ₘ, more preferably having the structure (HE)ₘ or (EH)ₘ, more preferably (EH)ₘ and with m being most preferably of from 2 to 10, more preferably of from 2 to 5, more preferably 3.

Thus, the following structure (1b_1) is particularly preferred:

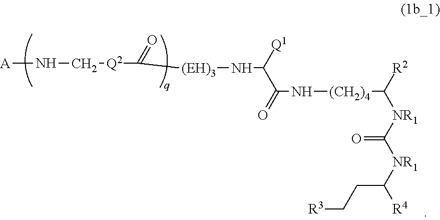

As outlined above, the amino acids E and H have preferably L-configuration.

### Preferred Embodiment 1bb

According to a further preferred embodiment, (m1+m2)n>1, wherein m1 is preferably 0 or 1 and m2 is preferably 0 or 1.

More preferably, (m1+m2)m is of from 2 to 10, more preferably of from 4 to 8, more preferably 6, with m1 being preferably 0 or 1 and m2 being preferably 0 or 1. It is to be understood that this includes e.g. combination of Y¹ and Y², such as e.g. ((Y₁)₁(Y²)₀)₆, ((Y¹)₀(Y²)₁)₆ as well as ((Y¹)₁(Y²)₁)₃.

According to embodiment (1bb), m1 is preferably 1 and m2 is 0, and m is preferably of from 2 to 10, more preferably of from 2 to 5, more preferably 3.

Y¹ is preferably a negatively charged or a positively charged amino acid, more preferably, Y¹ is histidine (H) or glutamic acid (E), more preferably histidine.

More preferably, according to embodiment (1b), (n1+n2)n+(p1+p2)p=0. Thus, according to this preferred embodiment, the compound has preferably the structure (1bb)

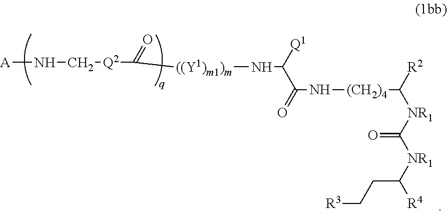

with the building block ((Y¹)ₘ₁)ₘ, more preferably having the structure (H)ₘ or (H)ₘ, with m being most preferably of from 2 to 10, more preferably of from 2 to 5, more preferably 3.

Thus, the following structure (1bb_1) is preferred:

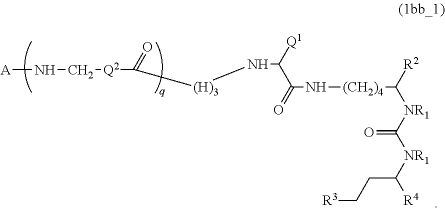

Thus, the following structure (1bb_2) is preferred:

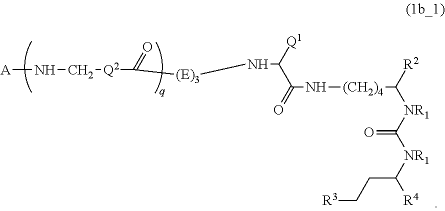

As outlined above, the amino acids E and H have preferably L-configuration.

### Preferred Embodiment 1c

According to a third preferred embodiment, (p1+p2)p>1, wherein p1 is preferably 0 or 1 and p2 is preferably 0 or 1.

More preferably, (p1+p2)p is of from 2 to 10, more preferably of from 4 to 8, more preferably 6, with p1 being preferably 0 or 1 and p2 being preferably 0 or 1. It is to be understood that this includes e.g. combination of Z¹ and ZY², such as e.g. ((Z¹)₁(Z²)₀)₆, ((Z¹)₀(Z²)₁)₆ as well as ((Z¹)₁(Z²)₁)₃.

According to embodiment (1c), p1 and p2 are preferably both 1 and p is of from 2 to 10, more preferably of from 2 to 5, more preferably 3.

Preferably, at least one of Z¹ or Z² in this embodiment is a negatively charged, and at least one of Z¹ and Z² is a positively charged amino acid.

More preferably, at least one of Z¹ and Z² is histidine (H) and at least one of Z¹ and Z² is glutamic acid (E). Even more preferably, the building block ((Z¹)ₚ₁(Z²)ₚ₂)ₚ, has the structure (HE)ₚ or (EH)ₚ, preferably (EH)ₚ.

More preferably, according to embodiment (1c), (n1+n2)n+(m1+m2)m=0. Thus, according to this preferred embodiment, the compound has preferably the structure (1c)

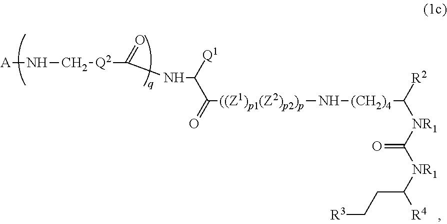

with the building block ((Z¹)ₚ₁(Z²)ₚ₂)ₚ, more preferably having the structure (HE)ₚ or (EH)ₚ, more preferably (EH)ₚ and with p being most preferably of from 2 to 10, more preferably of from 2 to 5, more preferably 3.

Thus, the following structure (1c_1) is particularly preferred:

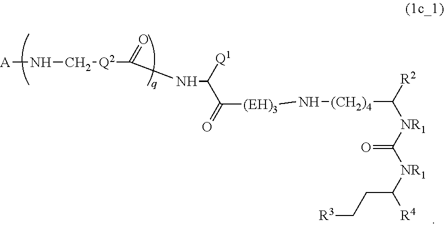

As outlined above, the amino acids E and H have preferably L-configuration.

### Preferred Embodiment 1cc

According to a further preferred embodiment, (p1+p2)p>1, wherein p1 is preferably 0 or 1 and p2 is preferably 0 or 1.

More preferably, (p1+p2)p is of from 2 to 10, more preferably of from 4 to 8, more preferably 6, with p1 being preferably 0 or 1 and p2 being preferably 0 or 1. It is to be understood that this includes e.g. combination of Z¹ and ZY², such as e.g. ((Z¹)₁(Z²)₀)₆, ((Z¹)₀(Z²)₁)₆ as well as ((Z¹)₁(Z²)₁)₃.

According to embodiment (1cc), p1 is preferably 1 and p2 is 0, and p is preferably of from 2 to 10, more preferably of from 2 to 5, more preferably 3.

Z¹ is preferably a negatively charged or a positively charged amino acid, more preferably, Z¹ is histidine (H) or glutamic acid (E), more preferably histidine.

More preferably, according to embodiment (1c), (n1+n2)n+(m1+m2)m=0. Thus, according to this preferred embodiment, the compound has preferably the structure (1cc)

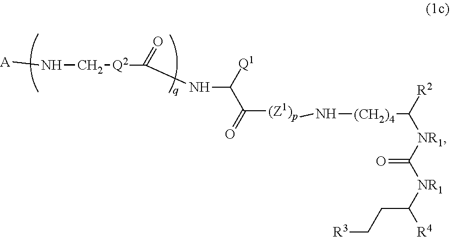

with the building block (Z¹)ₚ, more preferably having the structure (H)ₚ or (E)ₚ, with p being most preferably of from 2 to 10, more preferably of from 2 to 5, more preferably 3.

Thus, the following structure (1cc_1) is preferred:

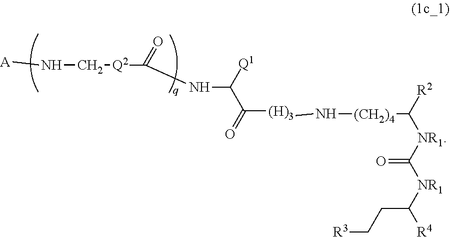

Further, the following structure (1cc_2) is preferred:

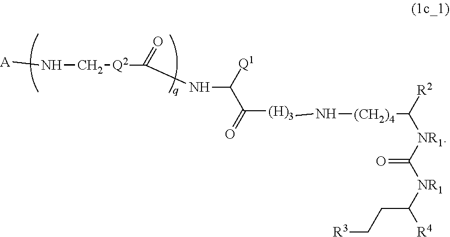

As outlined above, the amino acids E and H have preferably L-configuration.

R¹, R², R³ and R⁴

R¹ is H or —CH₃, preferably H.

R², R³ and R⁴ are independently of each other, selected from the group consisting of —CO₂H, —SO₂H, —SO₃H, —OSO₃H, —PO₂H, —PO₃H and —OPO₃H₂. More preferably, R², R³ and R⁴ are CO₂H.

Q1

Q¹ is preferably selected from the group consisting of alkylaryl, arylalkyl, aryl, alkylheteroaryl, heteroarylalkyl and heteroaryl,

The term “aryl”, as used in this context of the invention, means optionally substituted, 5- and 6-membered aromatic rings, and substituted or unsubstituted polycyclic aromatic groups (aryl groups), for example tricyclic or bicyclic aryl groups. Optionally substituted phenyl groups or naphthyl groups may be mentioned as examples. Polycyclic aromatic groups can also contain non-aromatic rings.

The term “alkylaryl” as used in this context of the invention refers to aryl groups in which at least one proton has been replaced with an alkyl group (Alkyl-aryl-).

The term “arylalkyl” as used in this context of the invention refers to aryl groups linked via an alkyl group (Aryl-alkyl-).

The term “heteroaryl”, as used in this context of the invention, means optionally substituted, 5- and 6-membered aromatic rings, and substituted or unsubstituted polycyclic aromatic groups, for example tricyclic or bicyclic aryl groups, containing one or more, for example 1 to 4, such as 1, 2, 3, or 4, heteroatoms in the ring system. If more than one heteroatom is present in the ring system, the at least two heteroatoms that are present can be identical or different. Suitable heteroaryl groups are known to the skilled person. The following heteroaryl residues may be mentioned, as non limiting examples: benzodioxolyl, pyrrolyl, furanyl, thiophenyl, thiazolyl, isothiaozolyl, imidazolyl, triazolyl, tetrazolyl, pyrazolyl, oxazolyl, isoxazolyl, pyridinyl, pyrazinyl, pyridazinyl, benzoxazolyl, benzodioxazolyl, benzothiazolyl, benzoimidazolyl, benzothiophenyl, methylenedioxyphenylyl, napthridinyl, quinolinyl, isoqunilyinyl, indolyl, benzofuranyl, purinyl, benzofuranyl, deazapurinyl, pyridazinyl and indolizinyl.

The term “alkylheteroaryl” as used in this context of the invention refers to heteroaryl groups in which at least one proton has been replaced with an alkyl group (Alkyl-Heteroaryl-).

The term “heteroarylalkyl” as used in this context of the invention refers to heteroaryl groups linked via an alkyl group (Heteroaryl-alkyl-).

The term “cycloalkyl” means, in the context of the invention, optionally substituted, cyclic alkyl residues, wherein they can be monocyclic or polycyclic groups. Optionally substituted cyclohexyl may be mentioned as a preferred example of a cycloalkyl residue.

The term “heterocycloalkyl”, as used in this context of the invention refers to optionally substituted, cyclic alkyl residues, which have at least one heteroatom, such as O, N or S in the ring, wherein they can be monocyclic or polycyclic groups.

The terms “substituted cycloalkyl residue” or “cycloheteroalkyl”, as used in this context of the invention refers, mean cycloalkyl residues or cycloheteroalkyl residues, in which at least one H has been replaced with a suitable substituent.

Preferably, Q1 comprises a residue selected from the group consisting of naphtyl, phenyl, biphenyl, indolyl, benzothiazolyl, naphtylmethyl, phenylmethyl, biphenylmethyl, indolylmethyl and benzothiazolylmethyl, more preferably Q¹ is selected from the group consisting of:

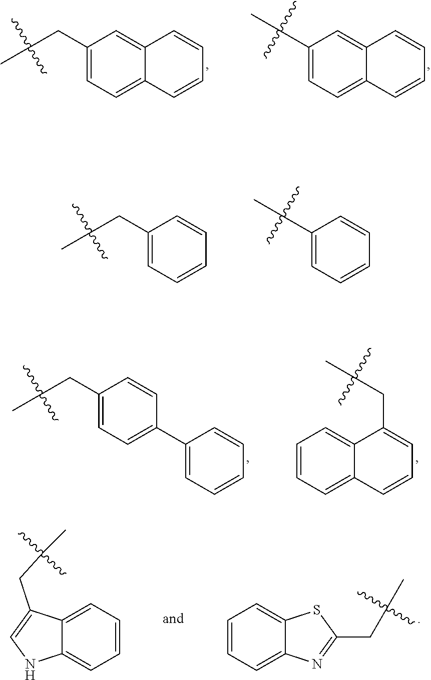

wherein Q¹ is most preferably

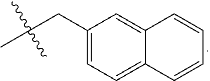 [SMILES: *Cc1ccc2ccccc2c1]

Thus, the compound preferably has the structure:

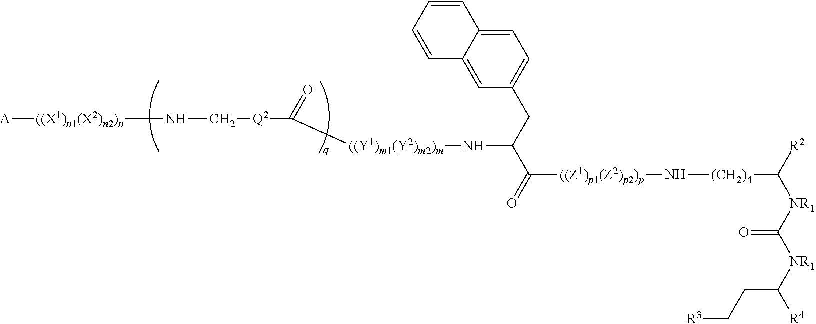

more preferably, a structure selected from the following group:

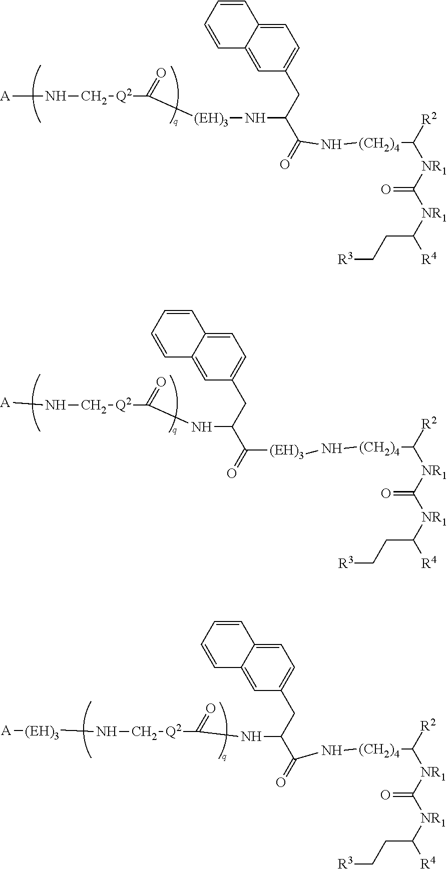

Q2

As described above, Q² is preferably selected from the group consisting of aryl, alkylaryl, arylalkyl, cycloalkyl, heterocycloalkyl, heteroaryl, heteroarylalkyl and alkylheteroaryl.

The term “aryl”, as used in this context of the invention refers to optionally substituted, 5- and 6-membered aromatic rings, and substituted or unsubstituted polycyclic aromatic groups (aryl groups), for example tricyclic or bicyclic aryl groups (—Ar—). Optionally substituted phenyl groups or naphthyl groups may be mentioned as examples. Polycyclic aromatic groups can also contain non-aromatic rings, the Aryl group in this context of the invention

The term “alkylaryl” as used in this context of the invention refers to aryl groups in which at least one proton has been replaced with an alkyl group (-alkyl-aryl-) and which are linked via to alkyl group to the —CH2- group and via the aryl group to the carbonyl group.

The term “arylalkyl” as used in this context of the invention refers to aryl groups linked via an alkyl group to the carbonyl group and via the aryl group to the —CH2- group (-aryl-alkyl-).

The term “heteroaryl” (-Heteraryl-, as used in this context of the invention, means optionally substituted, 5- and 6-membered aromatic rings, and substituted or unsubstituted polycyclic aromatic groups, for example tricyclic or bicyclic aryl groups, containing one or more, for example 1 to 4, such as 1, 2, 3, or 4, heteroatoms in the ring system. If more than one heteroatom is present in the ring system, the at least two heteroatoms that are present can be identical or different. Suitable heteroaryl groups are known to the skilled person. The following heteroaryl residues may be mentioned, as non limiting examples: benzodioxolyl, pyrrolyl, furanyl, thiophenyl, thiazolyl, isothiaozolyl, imidazolyl, triazolyl, tetrazolyl, pyrazolyl, oxazolyl, isoxazolyl, pyridinyl, pyrazinyl, pyridazinyl, benzoxazolyl, benzodioxazolyl, benzothiazolyl, benzoimidazolyl, benzothiophenyl, methylenedioxyphenylyl, napthridinyl, quinolinyl, isoqunilyinyl, indolyl, benzofuranyl, purinyl, benzofuranyl, deazapurinyl, pyridazinyl and indolizinyl.

The term “alkylheteroaryl” as used in this context of the invention refers to aryl groups in which at least one proton has been replaced with an alkyl group (-alkyl-heteroaryl-) and which are linked via to alkyl group to the —CH2- group and via the heteroaryl group to the carbonyl group.

The term “heteroarylalkyl” as used in this context of the invention refers to heteroaryl groups linked via an alkyl group to the carbonyl group and via the heteroaryl group to the —CH2- group (-aryl-alkyl-).

The term “cycloalkyl” (-cycloalkyl-) means, in the context of the invention, optionally substituted, cyclic alkyl residues, wherein they can be monocyclic or polycyclic groups.

Optionally substituted cyclohexyl may be mentioned as a preferred example of a cycloalkyl residue.

The term “heterocycloalkyl”, as used in this context of the invention refers to optionally substituted, cyclic alkyl residues, which have at least one heteroatom, such as O, N or S in the ring, wherein they can be monocyclic or polycyclic groups.

The terms “substituted cycloalkyl residue” or “cycloheteroalkyl”, as used in this context of the invention refers, mean cycloalkyl residues or cycloheteroalkyl residues, in which at least one H has been replaced with a suitable substituent.

Preferably, Q² is an aryl group or cycloalkyl group, more preferably

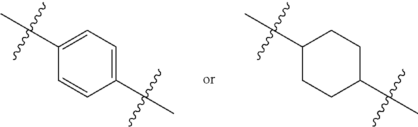 [SMILES: *c1ccc(*)cc1 ; *C1CCC(*)CC1]

most preferably.

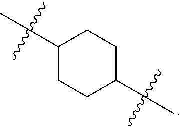 [SMILES: *C1CCC(*)CC1]

It is to be understood that any stereoisomers of Q² are possibly and included. In case Q² is

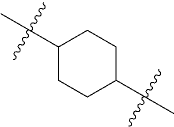 [SMILES: *C1CCC(*)CC1]

it is to be understood that this includes the cis as well as the trans isomer, with the trans isomer being particularly preferred.

Integer q is an integer of from 0-3, most preferably, q is 0 or 1, more preferably 1.

Chelator Residue A

A is a chelator residue derived from a chelator selected from the group consisting of 1,4,7,10-tetraazacyclododecane-N,N′,N″,N′″-tetraacetic acid (=DOTA), N,N″-bis[2-hydroxy-5-(carboxyethyl)benzyl]ethylenediamine-N,N″-diacetic acid, 1,4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7, 10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid] (NOPO), 3,6,9, 15-tetraazabicyclo[9.3.1.]pentadeca-1(15),11,13-triene-3,6,9-triacetic acid (=PCTA), N′-{5-[Acetyl(hydroxy)amino]pentyl}-N-[5-({4-[(5-aminopentyl)(hydroxy)amino]-4-oxobutanoyl}amino)pentyl]-N-hydroxysuccinamide (DFO), Diethylenetriaminepentaacetic acid (DTPA), Trans-cyclohexyl-diethylenetriaminepentaacetic acid (CHX-DTPA), 1-oxa-4,7, 10-triazacyclododecane-4,7, 10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl-DTPA (MX-DTPA)

The term “a chelator residue” and typically also the term “chelator residue derived from a chelator selected from the group” is denoted to mean that the above mentioned chelators, thus typically the chelators defined in the “group”, have been linked, via a suitable functional group, preferably via a former carboxylic acid group of the chelator, to the N-terminal end of compound (I):

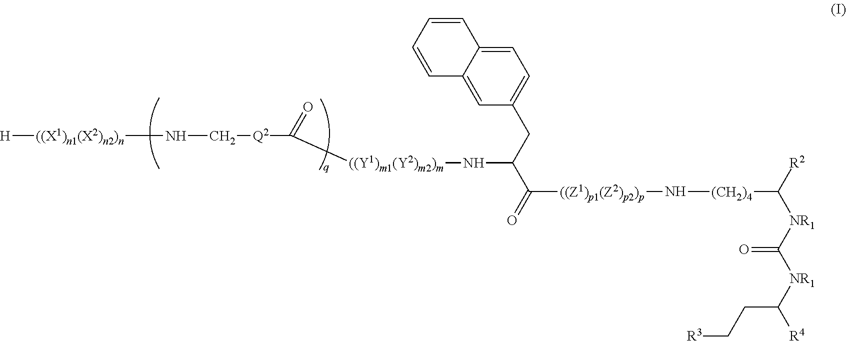

thereby forming an amide bond between the chelator and compound (I). If (n1+n2)n>0, the chelator is thus coupled to the N-Terminal group of the amino acid building block H—((X¹)ₙ₁(X²)ₙ₂)ₙ thereby forming an amide bond. If (n1+n2)n=0, and q is >0, the chelator is linked to the N-terminal NH group of the building block H—(NH—CH2-Q2-C(═O))_{q}—. If (n1+n2)n=0, and q is =0, and (m1+m2)m>0, the chelator is coupled to the N-Terminal group of the amino acid building block H—((Y)ₘ₁(Y²)ₘ₂)m thereby forming an amide bond. If (n1+n2)n=0, and q is =0, and (m1+m2)m=0, the chelator is coupled to the N-Terminal group of the amino acid building block H—(NH—CH(Q¹)—C(═O))— thereby forming an amide bond

Preferably, A is a chelator residue having a structure selected from the group consisting of

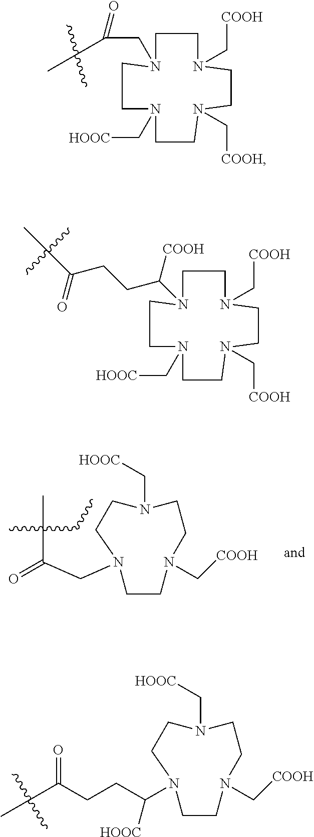

Most preferably, A has the structure

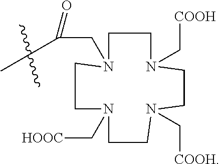 [SMILES: *C(=O)CN1CCN(CC(=O)O)CCN(CC(=O)O)CCN(CC(=O)O)CC1]

Complex

As described above, the present invention also relates to a complex comprising
(a) a radionuclide, and
(b) a compound, as described above or below, or a pharmaceutically acceptable salt or solvate thereof.

Typical pharmaceutically acceptable salts include those salts prepared by reaction of the compounds of the present invention with a pharmaceutically acceptable mineral or organic acid or an organic or inorganic base. Such salts are known as acid addition and base addition salts. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, napththalene-2-sulfonate, mandelate and the like. Preferred pharmaceutically acceptable acid addition salts are those formed with mineral acids such as hydrochloric acid and hydrobromic acid, and those formed with organic acids such as maleic acid and methanesulfonic acid. Salts of amine groups may also comprise quaternary ammonium salts in which the amino nitrogen carries a suitable organic group such as an alkyl, alkenyl, alkynyl, or aralkyl moiety. Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred. It should be recognized that the particular counter ion forming a part of any salt of this invention is usually not of a critical nature, so long as the salt as a whole is pharmacologically acceptable and as long as the counter ion does not contribute undesired qualities to the salt as a whole.

The term “pharmaceutically acceptable solvate” encompasses also suitable solvates of the compounds of the invention, wherein the compound combines with a solvent such as water, methanol, ethanol, DMSO, acetonitrile or a mixture thereof to form a suitable solvate such as the corresponding hydrate, methanolate, ethanolate, DMSO solvate or acetonitrilate.

The Radionuclide

Depending on whether the compounds of the invention are to be used as radio-imaging agents or radio-pharmaceuticals different radionuclides are complexed to the chelator.

The complexes of invention may contain one or more radionuclides, preferably one radionuclide. These radionuclides are preferably suitable for use as radio-imaging agents or as therapeutics for the treatment of proliferating cells, for example, PSMA expressing cancer cells, in particular PSMA-expressing prostate cancer cells. According to the present invention they are called “metal complexes” or “radiopharmaceuticals”.

Preferred imaging methods are positron emission tomography (PET) or single photon emission computed tomography (SPECT).

Preferably, the at least one radionuclide is selected from the group consisting ⁸⁹Zr, ⁴⁴Sc, ¹¹¹In, ⁹⁰Y, ⁶⁶Ga, ⁶⁷Ga, ⁶⁸Ga, ¹⁷⁷Lu, ^{99m}Tc, ⁶⁰Cu, ⁶¹Cu, ⁶²Cu, ⁶⁴Cu, ⁶⁶Cu, ⁶⁷Cu, ¹⁴⁹Tb, ¹⁵²Tb, ¹⁵⁵Tb, ¹⁵³Sm, ¹⁶¹Tb, ¹⁵³Gd, ¹⁵⁵Gd, ¹⁵⁷Gd, ²¹³Bi, ²²⁵Ac, ²³⁰U, ²²³Ra, ¹⁶⁵Er, ⁵²Fe, ⁵⁹Fe and radionuclides of Pb (such as ²⁰³Pb and ²¹²Pb, ²¹¹Pb, ²¹³Pb, ²¹⁴Pb, ²⁰⁹Pb, ¹⁹⁸Pb, ¹⁹⁷Pb).

More preferably, the at least one radionuclide is selected from the group consisting ⁹⁰Y, ⁶⁸Ga, ¹⁷⁷Lu, ²²⁵Ac, and ²¹³Bi. More preferably, the radionuclide is ¹⁷⁷Lu or ²²⁵Ac.

Preferably, the radionuclide has a half-life of at least 30 min, more preferably of at least 1 h, more preferably at least 12 h, even more preferably at least 1d, most preferably at least 5 d; also preferably, the radionuclide has a half-life of at most 1 year, more preferably at most 6 months, still more preferably at most 1 month, even more preferably at most 14 d. Thus, preferably, the radionuclide has a half-life of from 30 min to 1 year, more preferably of 12 h to 6 months, even more preferably of from 1 d to 1 month, most preferably of from 5 d to 14 d.

Preferably, the radionuclide is an α- and/or β-emitter, i.e. the radionuclide preferably emits α-particles (α-emitter) and/or β-radiation (β-emitter).

Preferably, in case the radionuclide is an α-emitter, the α-particle has an energy of from 1 to 10 MeV, more preferably of from 2 to 8 MeV, most preferably of from 4 to 7 MeV.

Preferably, in case the radionuclide is a β-emitter, the β-radiation has an energy of from 0.1 to MeV, more preferably of from 0.25 to 5 MeV, most preferably of from 0.4 to 2 MeV.

Preferred radionuclides emitting β-radiation are selected from the group consisting of ⁹⁰Y, ¹⁷⁷Lu, ⁵⁹Fe, 66Cu, ⁶⁷Cu, ¹⁶¹Tb, ¹⁵³Sm, ²¹²Pb, 211 Pb, ²¹³Pb, ²¹⁴Pb, ²⁰⁹Pb Very preferred radionuclides emitting β-radiation are ¹⁷⁷Lu or ⁹⁰Y, most preferably ¹⁷⁷Lu. Preferably in this case the use is diagnosis or therapy.

Preferred radionuclides emitting α-radiation are e.g. selected from the group consisting of ²¹³Bi, ²²⁵Ac, ¹⁴⁹Tb, ²³⁰U and ²²³Ra. ²¹³Bi, ²³⁰U, more preferably the radionuclide is ²²⁵Ac and/or ²¹³Bi. A very preferred radionuclide emitting α-radiation is e.g. ²²⁵Ac. Preferably in this case the use is therapy.

According to a further embodiment, the radionuclide is a positron emitter. In this case the radionuclide is preferably selected from the group consisting ⁸⁹Zr, ⁴⁴Sc, ⁶⁶Ga, ⁶⁸Ga and ⁶⁴Cu. In this case, the use is preferably PET diagnosis.

According to a further preferred embodiment, radionuclide is a gamma emitter. In this case the radionuclide is preferably selected from the group consisting ¹¹¹In, ⁶⁷Ga, ^{99m}Tc, ¹⁵⁵Tb, ¹⁶⁵Er and ²⁰³Pb. In this case, the use preferably is SPECT diagnosis.

According to a further preferred embodiment, the radionuclide emits Auger electrons, and preferably decays by electron capture. In this case, the radionuclide is preferably selected from the group consisting of ⁶⁷Ga, ¹⁵⁵Tb, ¹⁵³Gd, ¹⁶⁵Er and ²⁰³Pb. In this case, the use is preferably therapy.

Pharmaceutical Composition

As described above, the present invention also relates to a pharmaceutical composition comprising a compound as described above or below, or a complex as described above or below. It is to be understood that the pharmaceutical compositions preferably comprise therapeutically effective amounts of the compound and/or the complex, respectively. The pharmaceutical composition may further comprise at least one organic or inorganic solid or liquid and/or at least one pharmaceutically acceptable carrier.

The terms “medicament” and “pharmaceutical composition”, as used herein, relate to the compounds and/or complexes of the present invention and optionally one or more pharmaceutically acceptable carrier, i.e. excipient. The compounds of the present invention can be formulated as pharmaceutically acceptable salts; salts have been described herein above. The pharmaceutical compositions are, preferably, administered locally (e.g. intra-tumorally), topically or systemically. Suitable routes of administration conventionally used for drug administration are oral, intravenous, or parenteral administration as well as inhalation. A preferred route of administration is parenteral administration. A “parenteral administration route” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Preferably, administration is by intravenous administration or infusion. However, depending on the nature and mode of action of a compound, the pharmaceutical compositions may be administered by other routes as well.

Moreover, the compounds can be administered in combination with other drugs either in a common pharmaceutical composition or as separated pharmaceutical compositions wherein said separated pharmaceutical compositions may be provided in form of a kit of parts. The compounds are, preferably, administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables.

The excipient(s) must be acceptable in the sense of being compatible with the other ingredients of the formulation and, within the scope of sound medical judgment, suitable for use in contact with the tissues of a patient without excessive toxicity, irritation, allergic response, or other problem or complication, commensurate with a reasonable benefit/risk ratio. Preferably, an excipient is being not deleterious to the recipient thereof. The excipient employed may be, for example, a solid, a gel or a liquid carrier. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa. The diluent(s) is/are selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, non-immunogenic stabilizers and the like. When solutions for infusion or injection are used, they are preferably aqueous solutions or suspensions, it being possible to produce them prior to use, e.g. from lyophilized preparations which contain the active substance as such or together with a carrier, such as mannitol, lactose, glucose, albumin and the like. The readymade solutions are sterilized and, where appropriate, mixed with excipients, e.g. with preservatives, stabilizers, emulsifiers, solubilizers, buffers and/or salts for regulating the osmotic pressure. The sterilization can be obtained by sterile filtration using filters having a small pore size according to which the composition can be lyophilized, where appropriate. Small amounts of antibiotics can also be added to ensure the maintenance of sterility.

A therapeutically effective dose refers to an amount of the compounds to be used in a pharmaceutical composition of the present invention which prevents, ameliorates or treats the symptoms accompanying a disease or condition referred to in this specification. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The dosage regimen will be determined by the attending physician and other clinical factors; preferably in accordance with any one of the above described methods. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment. Preferred doses are specified herein below. Progress can be monitored by periodic assessment. The pharmaceutical compositions and formulations referred to herein are administered at least once in order to treat or prevent a disease or condition recited in this specification. However, the said pharmaceutical compositions may be administered more than one time, for example from one to ten times. Preferably, the pharmaceutical compositions may be administered at a frequency of once every one to six months, more preferably once every two to four months. Specific pharmaceutical compositions are prepared in a manner well known in the pharmaceutical art and comprise at least one active compound referred to herein above in admixture or otherwise associated with a pharmaceutically acceptable carrier or diluent. For making those specific pharmaceutical compositions, the active compound(s) will usually be mixed with a carrier or the diluent, or enclosed or encapsulated in a capsule, sachet, cachet, paper or other suitable containers or vehicles. The resulting formulations are to be adapted to the mode of administration, i.e. in the forms of tablets, capsules, suppositories, solutions, suspensions or the like. Dosage recommendations shall be indicated in the prescribers or users instructions in order to anticipate dose adjustments depending on the considered recipient.

The term “patient”, as used herein, relates to a vertebrate, preferably a mammalian animal, more preferably a human, monkey, cow, horse, cat or dog. Preferably, the mammal is a primate, more preferably a monkey, most preferably a human).

The dosage of the compound according to formula (1) administered to a patient, preferably, is defined as a compound dosage, i.e. the amount of compound administered to the patient. Preferred diagnostic compound dosages are total doses of 1-10 nmol/patient; thus, preferably, the diagnostic compound dosage is of from 0.02 to 0.1 nmol/kg body weight. Preferred therapeutic compound dosages are total doses of 10 to 100 nmol/patient; thus, preferably, the therapeutic compound dosage is of from 0.2 to 1 nmol/kg body weight.

As will be understood by the skilled person, the dosage of the complex as specified herein, i.e. a complex comprising, preferably consisting of, a radionuclide and a compound according to formula (1), preferably is indicated as compound dosage as specified above, preferred dosages being the same as specified above. More preferably, the dosage of the complex is indicated as activity dosage, i.e. as the amount of radioactivity administered to the patient. Preferably, the activity dosage is adjusted such as to avoid adverse effects as specified elsewhere herein. Preferably, a patient-specific dose, preferably a patient-specific activity dosage, is determined taking into account relevant factors as specified elsewhere herein, in particular taking into account therapeutic progress and/or adverse effects observed for the respective patient. Thus, preferably, the activity dosage is adjusted such that the organ-specific dose in salivary glands is at most 30 Sv, more preferably less than 20 Sv, still more preferably less than 10 Sv, most preferably less than 5 Sv.

The effective amount may be administered once (single dosage) with an activity dosage of from about 2 MBq to about 30 MBq, preferably 4 to 30 Mbq, more preferably 6 to 30 Mbq, more preferably 8 to 30 Mbq, more preferably 10 to 30 Mbq, more preferably 15 to 30 Mbq, preferably 20 to 30 Mbq to the patient. Thus, a preferred therapeutic dose in such case is of from 2 MBq to about 30 MBq/patient, preferably 4 to 30 Mbq/patient, more preferably 6 to 30 Mbq/patient, more preferably 8 to 30 Mbq/patient, more preferably 10 to 30 Mbq/patient, more preferably 15 to 30 Mbq/patient, preferably 20 to 30 Mbq/patient. Preferably said activity dosage ranges from about 10 to 30 MBq per administration, such as for example about 10, 11, 12, 13, 14, 15, 16, 17, 18, 19, 20, 21, 22, 23, 24, 25, 26, 27, 28, 29 or 30 MBq, or any range between any two of the above values. However, as specified herein below, depending on the type of radiation emitted by the radionuclide and/or on the application, higher or lower doses may be envisaged. The phrases “effective amount” or “therapeutically-effective amount” as used herein mean that amount of a compound, material, or composition comprising a compound of the invention, or other active ingredient which is effective for producing some desired therapeutic effect in at least a sub-population of cells in a patient at a reasonable benefit/risk ratio applicable to any medical treatment. A therapeutically effective amount with respect to a compound of the invention means that amount of therapeutic agent alone, or in combination with other therapies, that provides a therapeutic benefit in the treatment or prevention of a disease. Used in connection with a compound of the invention, the term can encompass an amount that improves overall therapy, reduces or avoids symptoms or causes of disease, or enhances the therapeutic efficacy of or synergies with another therapeutic agent.

According to a preferred embodiment, the radionuclide is a β-emitter as specified herein above, more preferably is ¹⁷⁷Lu and the use is diagnosis; in such case, the activity dosage of the complex preferably is at least 100 kBq/kg body weight, more preferably at least 500 kBq/kg body weight, most preferably at least 1 MBq/kg body weight. More preferably the radionuclide is a β-emitter as specified herein above, more preferably is ¹⁷⁷Lu and the use is therapy, preferably therapy of prostate carcinoma as specified elsewhere herein; in such case, the activity dosage of the complex preferably is at least 25 MBq/kg body weight, more preferably at least 50 MBq/kg body weight, most preferably at least 80 MBq/kg body weight. Thus, a preferred therapeutic dose in such case is of from 2 to 10 Gbq/patient, more preferably of from 4 to 8 GBq/patient, most preferably is about 6 GBq/patient.

More preferably, the radionuclide is an α-emitter as specified herein above, more preferably is ²²⁵Ac and the use is therapy, preferably therapy of prostate carcinoma as specified elsewhere herein; in such case, the activity dosage of the complex is preferably in the range of from 25 kBq/kg to about 500 kBq/kg of body weight of said patient, more preferably, the activity dosage of the complex is at least 75 kBq/kg body weight, more preferably at least 100 kBq/kg body weight, still more preferably at least 150 kBq/kg body weight, most preferably at least 200 kBq/kg body weight. Thus, preferably, in such case, the activity dosage of the complex is of from 75 to 500 kBq/kg body weight, more preferably of from 100 to 400 kBq/kg body weight, still more preferably of from 150 to 350 kBq/kg body weight, most preferably of from 200 to 300 kBq/kg body weight.

The present invention also relates to a compound as described above or below, a complex as described above or below, or a pharmaceutical composition as described herein above, for use in diagnosis, preferably for diagnosing a cell proliferative disease or disorder, in particular prostate cancer and/or metastases thereof. Further, the present invention also relates to a compound as described above or below a complex as described above or below, or a pharmaceutical composition as described above or below, for use in medicine, preferably for treating or preventing a cell proliferative disease or disorder, in particular prostate cancer and/or metastases thereof.

The term “diagnosing”, as used herein, refers to assessing whether a subject suffers from a disease or disorder, preferably cell proliferative disease or disorder, or not. As will be understood by those skilled in the art, such an assessment, although preferred to be, may usually not be correct for 100% of the investigated subjects. The term, however, requires that a, preferably statistically significant, portion of subjects can be correctly assessed and, thus, diagnosed. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2, 0.1, or 0.05. As will be understood by the skilled person, diagnosing may comprise further diagnostic assessments, such as visual and/or manual inspection, determination of tumor biomarker concentrations in a sample of the subject, X-ray examination, and the like. The term includes individual diagnosis of as well as continuous monitoring of a patient. Monitoring, i.e. diagnosing the presence or absence of cell proliferative disease or the symptoms accompanying it at various time points, includes monitoring of patients known to suffer from cell proliferative disease as well as monitoring of subjects known to be at risk of developing cell proliferative disease. Furthermore, monitoring can also be used to determine whether a patient is treated successfully or whether at least symptoms of cell proliferative disease can be ameliorated over time by a certain therapy. Moreover, the term also includes classifying a subject according to a usual classification scheme, e.g. the T1 to T4 staging, which is known to the skilled person.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, as specified herein above. The term “preventing” and “prevention” refers to retaining health with respect to the diseases or disorders referred to herein for a certain period of time in a subject. It will be understood that the said period of time may be dependent on the amount of the drug compound which has been administered and individual factors of the subject discussed elsewhere in this specification. It is to be understood that prevention may not be effective in all subjects treated with the compound according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed herein above.

Preferably, treatment and/or prevention comprises administration of at least one compound according to formula (1) and/or at least one complex as specified elsewhere herein, more preferably at an activity dosage and/or compound dosage as specified above.

The term “cell proliferative disease”, as used herein, relates to a disease of an animal, including man, characterized by uncontrolled growth by a group of body cells (“cancer cells”). This uncontrolled growth may be accompanied by intrusion into and destruction of surrounding tissue and possibly spread of cancer cells to other locations in the body (metastasis). Preferably, also included by the term cancer is a relapse. Thus, preferably, the cancer is a solid cancer, a metastasis, or a relapse thereof. Preferably, the cell proliferative disease is an uncontrolled proliferation of cells comprising cells expressing PSMA.

Thus, preferably, the cell proliferative disease is a PSMA expressing cancer. The term “PSMA expressing cancer” refers to any cancer whose cancerous cells express Prostate Specific Membrane Antigen (PSMA). Preferably cancers (or cancer cells) that may be treated according to the invention are selected among prostate cancer, conventional renal cell cancers, cancers of the transitional cells of the bladder, lung cancers, testicular-embryonal cancers, neuroendocrine cancers, colon cancers, brain tumors and breast cancers, more preferably are selected among PSMA-positive prostate cancer, PSMA-positive renal cell cancers, PSMA-positive cancers of the transitional cells of the bladder, PSMA-positive lung cancers, PSMA-positive testicular-embryonal cancers, PSMA-positive neuroendocrine cancers, PSMA-positive colon cancers, PSMA-positive brain tumors, and PSMA-positive breast cancers. Whether a cancer is PSMA-positive can be established by the skilled person by methods known in the art, e.g. in vitro by immunostaining of a cancer sample, or in vivo e.g. by PSMA scintigraphy, preferably both as described in Kratochwil et al. (2017, J Nucl Med 58(10):1624. In particularly preferred aspects of the invention, said PSMA expressing cancer is prostate cancer or breast cancer, more preferably prostate cancer; and even more preferably advanced-stage prostate cancer. Thus, preferably, the cell proliferative disease is prostate cancer stage T2, more preferably stage T3, most preferably stage T4. Preferably, the cell proliferative disease is metastatic prostate cancer, more preferably is metastatic castration-resistant prostate cancer. Advantageously, it has been shown in the studies underlying the present invention that administration of the compounds and/or complexes of the present invention to a patient results in a reduced uptake of said compounds and/or complexes by the salivary and lacrimal glands, i.e. the patient's salivary and lacrimal glands, as compared to the uptake of e.g. the meanwhile commonly used PSMA-617. Due to the reduced uptake, adverse side effects on the salivary and/or lacrimal glands can be avoided and/or reduced. This is advantageous, because the adverse side effects on the salivary glands are considered as dosage-limiting (cf. Kratochwil et al. (2017, J Nucl Med 58(10):1624). Based on the finding of the present invention, larger amounts of compounds and/or complexes and in particular higher doses of radioactivity can be administered to a patient as compared to the compounds and complexes described in the art. Thus, the therapeutic window is broader than with the compounds presently in use. Also advantageously, the compounds of the present invention provide for improved diagnosis, since the co-labelling of irrelevant tissue and organs, in particular salivary glands, lacrimal glands and/or kidneys, is reduced.

Thus, the compounds and/or complexes of the present invention allow for the treatment of PSMA-expressing cancers, especially prostate cancer, and metastases thereof, and/or the diagnosis of PSMA-expressing cancers, especially prostate cancer, and metastases thereof, wherein adverse side effects on the patient's salivary glands and/or lacrimal glands are avoided and/or reduced. Thus, said treatment and/or diagnosis has less or less severe adverse side effects on the salivary glands and/or lacrimal glands or is preferably not accompanied by adverse side effects on the salivary glands and/or lacrimal glands at all. Preferably, the compounds of the present invention allow for reduction and/or avoidance of adverse side effects on the salivary glands and/or lacrimal glands while maintaining therapeutic efficacy essentially unchanged; thus, preferably, excretory properties of the compounds of the present invention are essentially unchanged compared to PSMA-617.

Accordingly, the compounds and/or complexes of the present invention allow for the treatment of PSMA-expressing cancers, especially prostate cancer, and metastases thereof, and/or the diagnosis of PSMA-expressing cancers, especially prostate cancer, and metastases thereof, wherein xerostomia is avoided.

Preferably the compound, as described above or below, or the complex, as described above or below, or the pharmaceutical composition, as described above or below, are used for in vivo imaging and radiotherapy. Suitable pharmaceutical compositions may contain a radio imaging agent, or a radiotherapeutic agent that has a radionuclide either as an element, i.e. radioactive iodine, or a radioactive metal chelate complex of the compound of formula (1a) and/or (1b) in an amount sufficient for imaging, together with a pharmaceutically acceptable radiological vehicle. The radiological vehicle should be suitable for injection or aspiration, such as human serum albumin; aqueous buffer solutions, e.g., tris(hydromethyl)-aminomethane (and its salts), phosphate, citrate, bicarbonate, etc; sterile water physiological saline; and balanced ionic solutions containing chloride and or dicarbonate salts or normal blood plasma cautions such as calcium potassium, sodium and magnesium.

The concentration of the imaging agent or the therapeutic agent in the radiological vehicle should be sufficient to provide satisfactory imaging. Appropriate dosages have been described herein above. The imaging agent or therapeutic agent should be administered so as to remain in the patient for about 1 hour to 10 days, although both longer and shorter time periods are acceptable. Therefore, convenient ampoules containing 1 to 10 mL of aqueous solution may be prepared.

Imaging may be carried out in a manner known to the skilled person, for example by injecting a sufficient amount of the imaging composition to provide adequate imaging and then scanning with a suitable imaging or scanning machine, such as a tomograph or gamma camera. In certain embodiments, a method of imaging a region in a patient includes the steps of: (i) administering to a patient a diagnostically effective amount of a compound complexed with a radionuclide; (ii) exposing a region of the patient to the scanning device; and (ii) obtaining an image of the region of the patient. In certain embodiments of the region imaged is the head or thorax. In other embodiments, the compounds and complexes of formula (1a) and/or (1b) target the PSMA protein.

Thus, in some embodiments, a method of imaging tissue such as spleen tissue, kidney tissue, or PSMA-expressing tumor tissue is provided including contacting the tissue with a complex synthesized by contacting a radionuclide and a formula (1a) and/or formula (1b) compound.

The amount of the compound of the present invention, or a formulation comprising a complex of the compound, or its salt, solvate, stereoisomer, or tautomer that is administered to a patient depends on several physiological factors. These factors are known by the physician, including the nature of imaging to be carried out, tissue to be targeted for imaging or therapy and the body weight and medical history of the patient to be imaged or treated using a radiopharmaceutical.

Accordingly in another aspect, the invention provides a method for treating a patient by administering to a patient a therapeutically effective amount of a complex, as described above or below, to treat a patient suffering from a cell proliferative disease or disorder. Specifically, the cell proliferative disease or disorder to be treated or imaged using a compound, pharmaceutical composition or radiopharmaceutical in accordance with this invention is a cancer, for example, prostate cancer and/or prostate cancer metastasis in e.g. lung, liver, kidney, bones, brain, spinal cord, bladder, etc.

The compounds of the invention may e.g. be synthesized in solution as well as on solid phase using e.g. standard peptide coupling procedures, such as Fmoc solid phase coupling procedures. Preferably, the chelator is coupled to the remaining part of the molecule in the last coupling step followed by a deprotection step and in case of solid phase chemistry, cleavage from the resin. However, other synthetic procedures are possible and known to the skilled person. A preferred synthesis of the compounds of the present invention is described in detail in the example section

By way of example, the particularly preferred compounds of the invention are shown in Table 1:

TABLE 1
Preferred compounds, by way of example

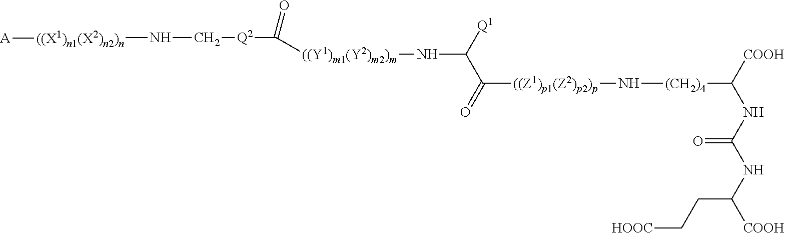

| A | —(X¹)ₙ₁(X²)ₙ₂)ₙ— | Q2 | q | —(Y¹)ₘ₁(Y²)ₘ₂)ₘ— | Q1 | —(Z¹)ₚ₁(Z²)ₚ₂)ₚ— |
|---|---|---|---|---|---|---|
| 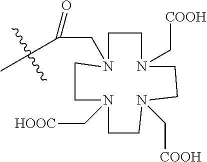 [SMILES: *C(=O)CN1CCN(CC(=O)O)CCN(CC(=O)O)CCN(CC(=O)O)CC1] | —(EH)₂—, —(EH)₃—, —(EH)₄—, —(EH)₅—, —(HE)₃—, —(E)₃—, or —(H)₃—, preferably —(EH)₃—, (HE)3 (EH)3 —(E)₃—, or —(H)₃—, in particular —(EH)₃— | -p-trans- cyclohexyl | 1 | m = 0 | —CH₂— naphtyl | p = 0 |
| 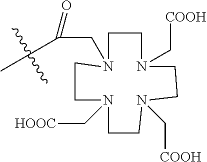 [SMILES: *C(=O)CN1CCN(CC(=O)O)CCN(CC(=O)O)CCN(CC(=O)O)CC1] | n = 0 | -p-trans- cyclohexyl | 1 | —(EH)₂—, —(EH)₃—, —(EH)₄—, —(EH)₅—, —(E)₃—, or —(H)₃—, preferably —(EH)₃—, —(E)₃—, or —(H)₃—, in particular —(EH)₃— | —CH₂— naphtyl | p = 0 |
| 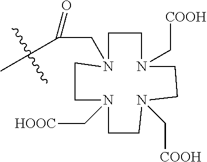 [SMILES: *C(=O)CN1CCN(CC(=O)O)CCN(CC(=O)O)CCN(CC(=O)O)CC1] | n = 0 | -p-trans- cyclohexyl | 1 | m = 0 | —CH₂— naphtyl | —(EH)₂—, —(EH)₃—, —(EH)₄—, —(EH)₅—, —(E)₃—, or —(H)₃—, preferably —(EH)₃—, —(E)₃—, or —(H)₃—, in particular —(EH)₃— |

Preferably, the compound has thus a structure selected from the group consisting of the following structures:

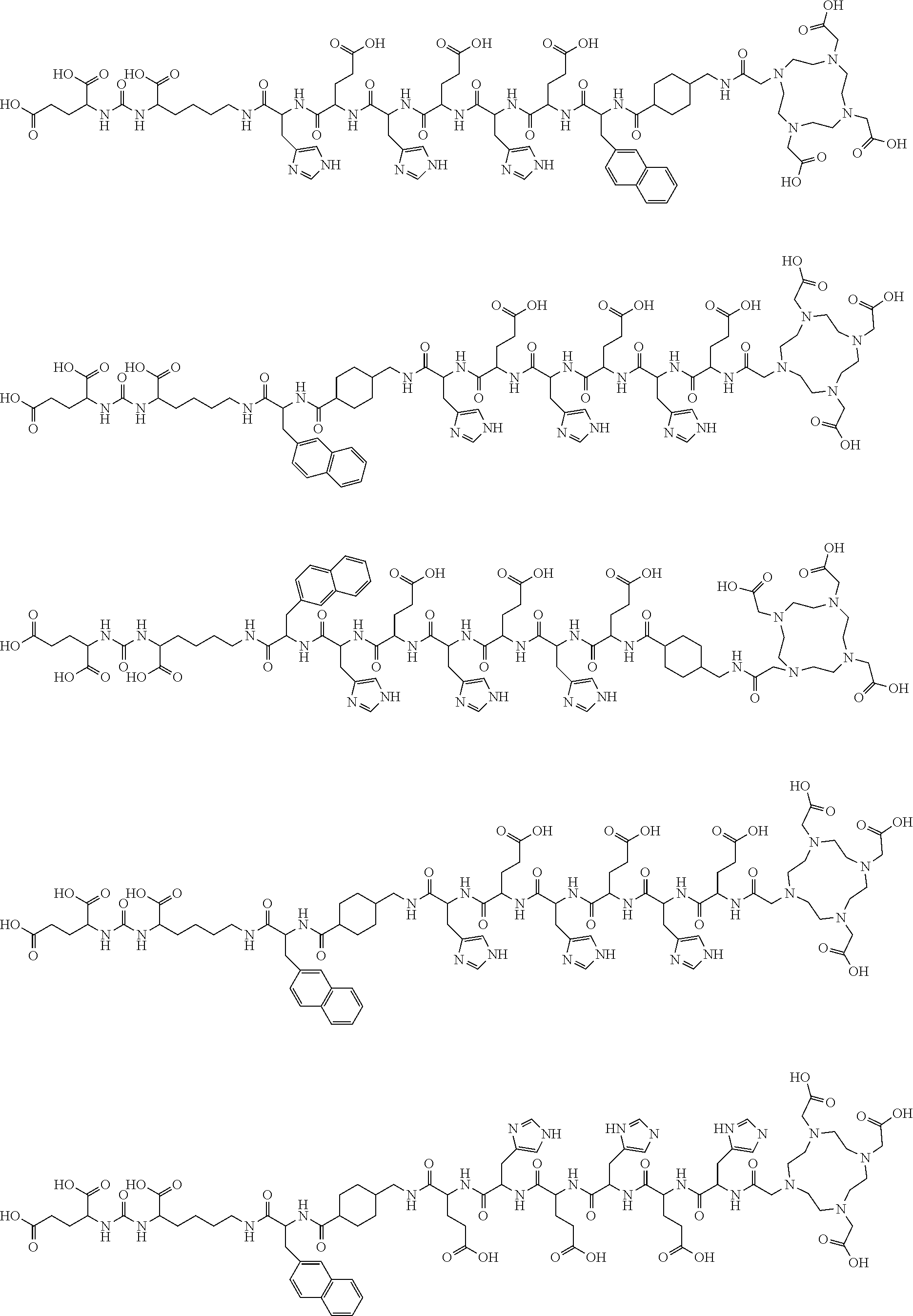

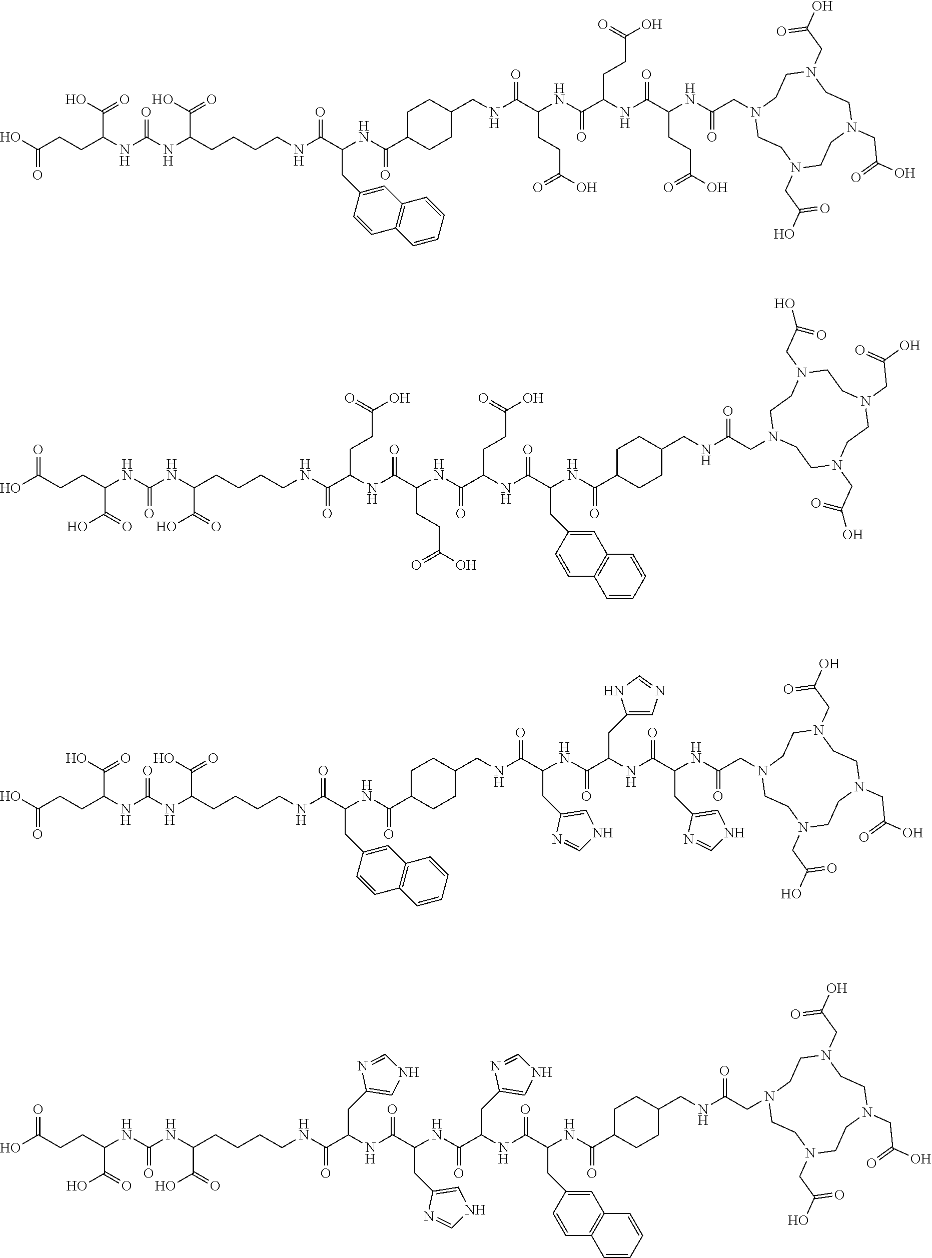

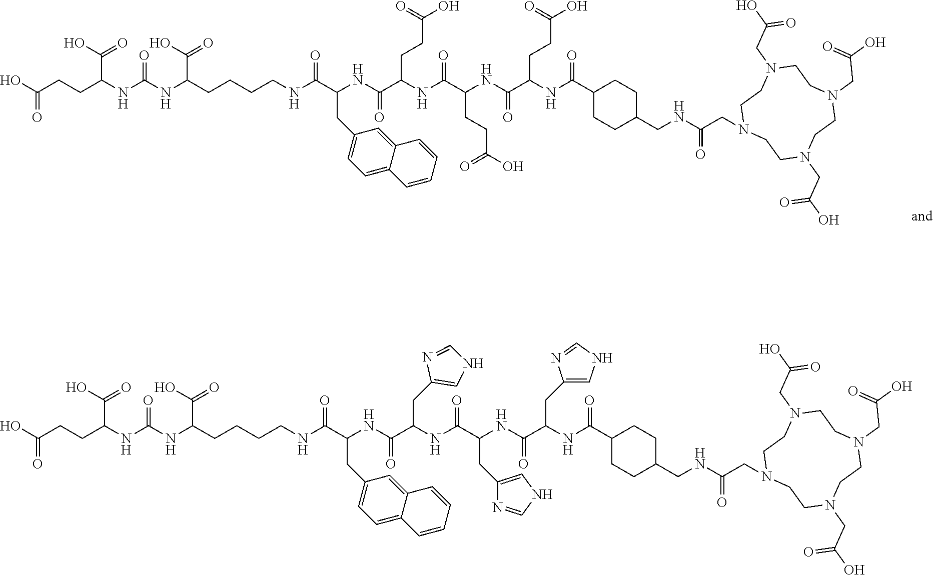 [SMILES: O=C(O)CCC(NC(=O)NC(CCCCNC(=O)C(Cc1ccc2ccccc2c1)NC(=O)C(CCC(=O)O)NC(=O)C(CCC(=O)O)NC(=O)C(CCC(=O)O)NC(=O)C1CCC(CNC(=O)CN2CCN(CC(=O)O)CCN(CC(=O)O)CCN(CC(=O)O)CC2)CC1)C(=O)O)C(=O)O ; O=C(O)CCC(NC(=O)NC(CCCCNC(=O)C(Cc1ccc2ccccc2c1)NC(=O)C(Cc1c[nH]cn1)NC(=O)C(Cc1c[nH]cn1)NC(=O)C(Cc1c[nH]cn1)NC(=O)C1CCC(CNC(=O)CN2CCN(CC(=O)O)CCN(CC(=O)O)CCN(CC(=O)O)CC2)CC1)C(=O)O)C(=O)O]

More preferably, the compound has a structure selected from the group consisting of the following structures:

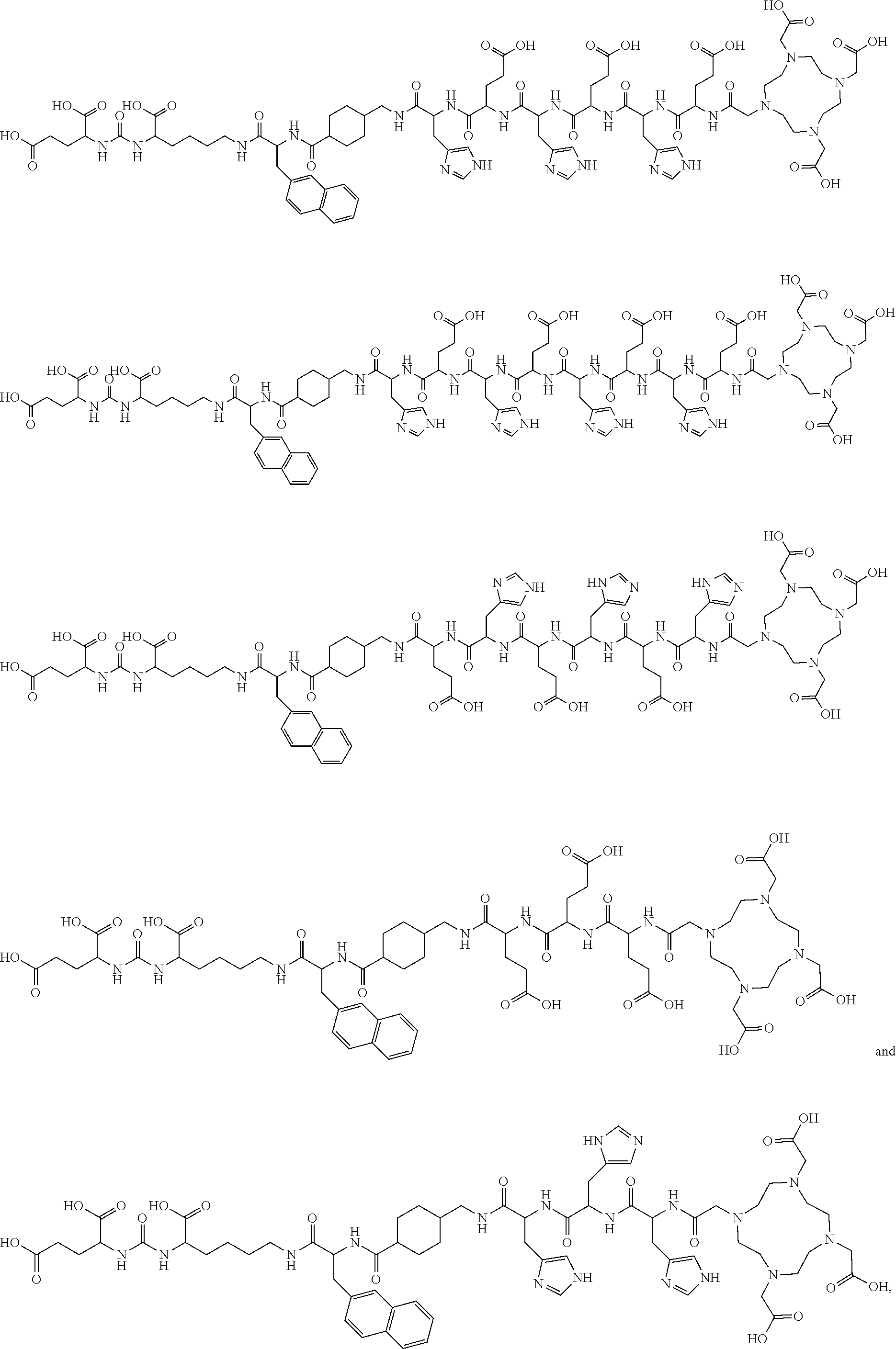

more preferably of the group consisting of

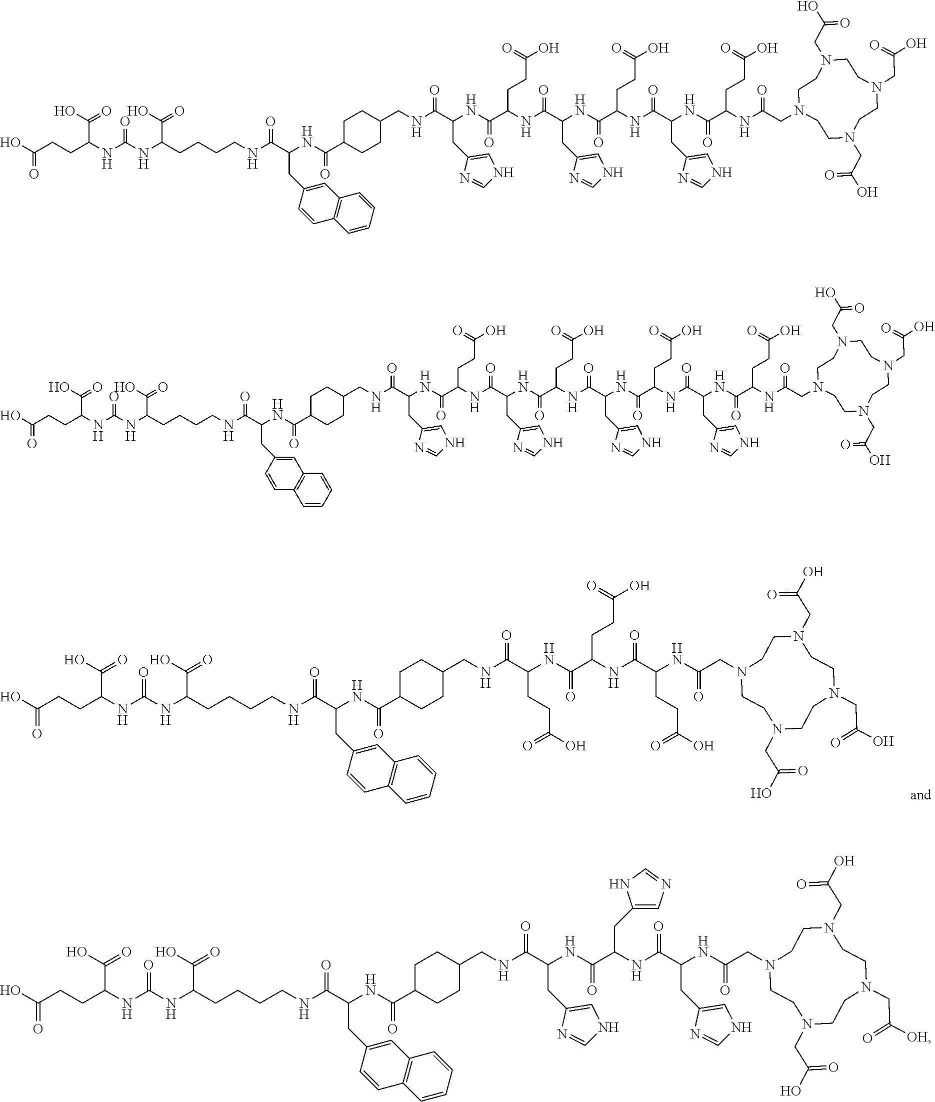

Particularly preferable, the compound has the structure:

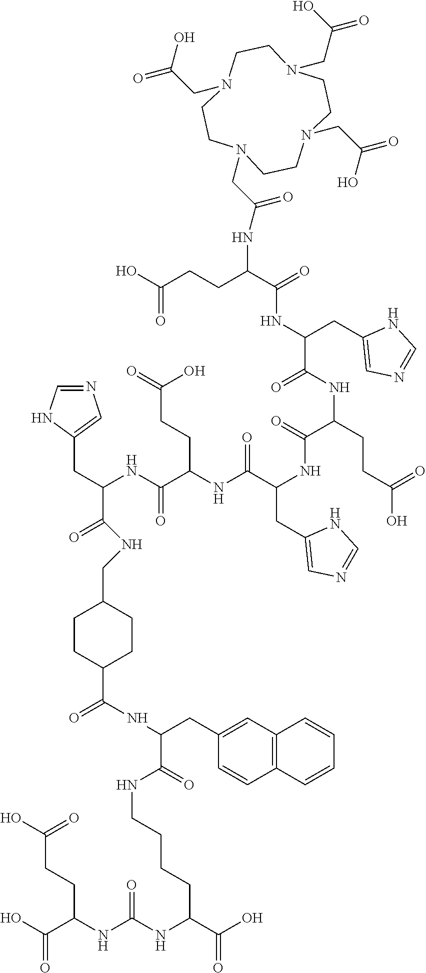 [SMILES: O=C(O)CCC(NC(=O)NC(CCCCNC(=O)C(Cc1ccc2ccccc2c1)NC(=O)C1CCC(CNC(=O)C(Cc2cnc[nH]2)NC(=O)C(CCC(=O)O)NC(=O)C(Cc2cnc[nH]2)NC(=O)C(CCC(=O)O)NC(=O)C(Cc2cnc[nH]2)NC(=O)C(CCC(=O)O)NC(=O)CN2CCN(CC(=O)O)CCN(CC(=O)O)CCN(CC(=O)O)CC2)CC1)C(=O)O)C(=O)O]

As outlined above, the amino acids E and H have preferably L-configuration. More preferably, the compound has thus a structure selected from the group consisting of the following structures:

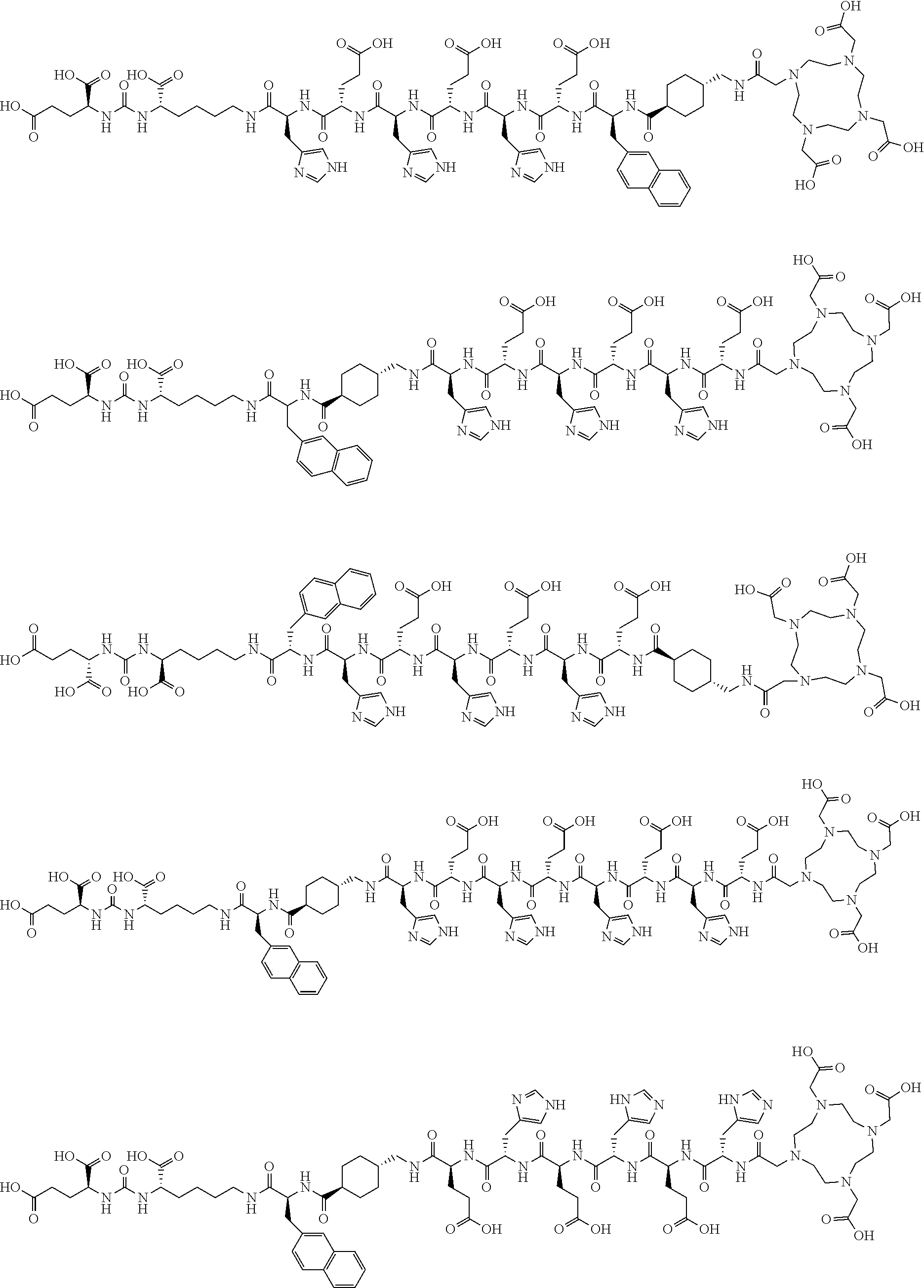

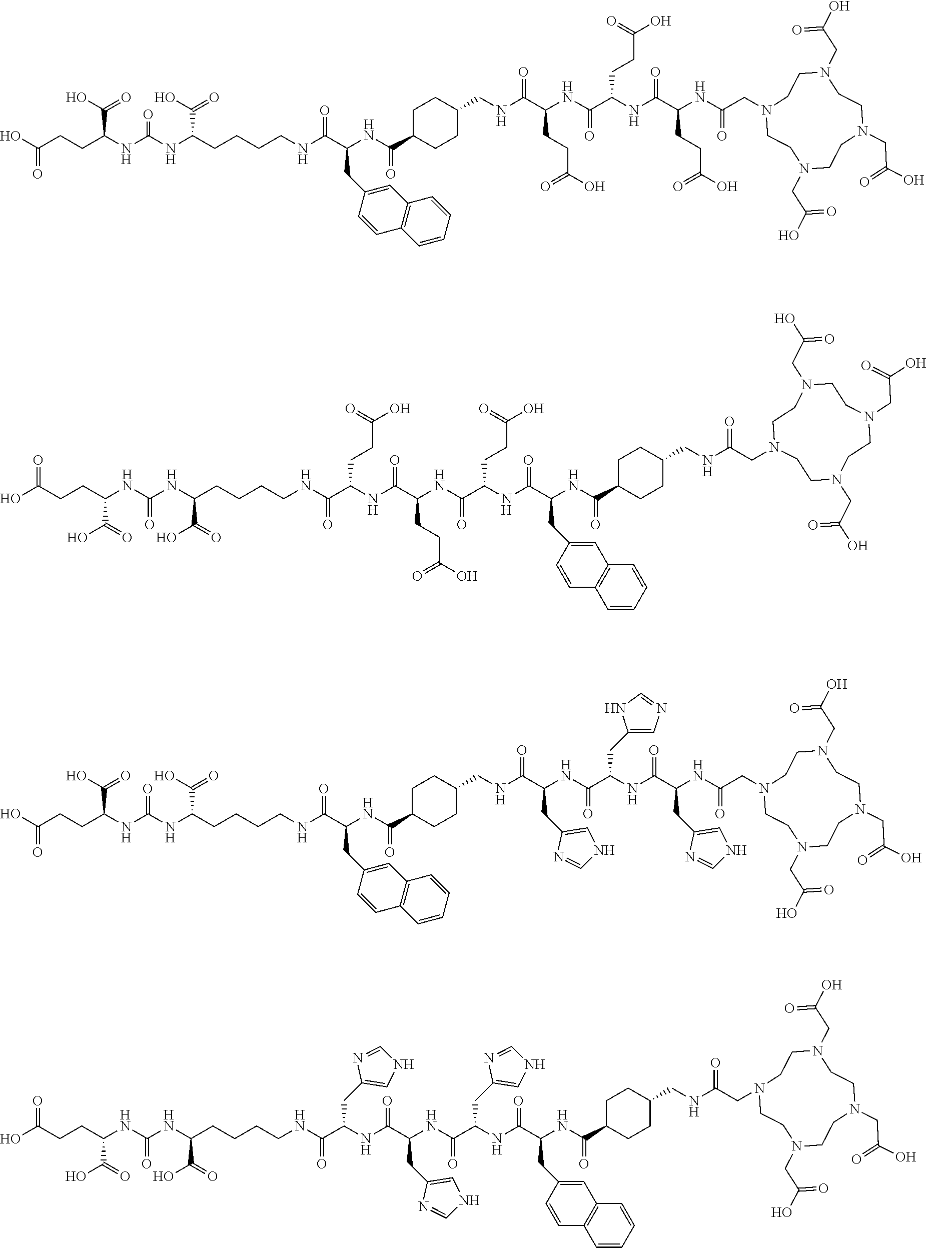

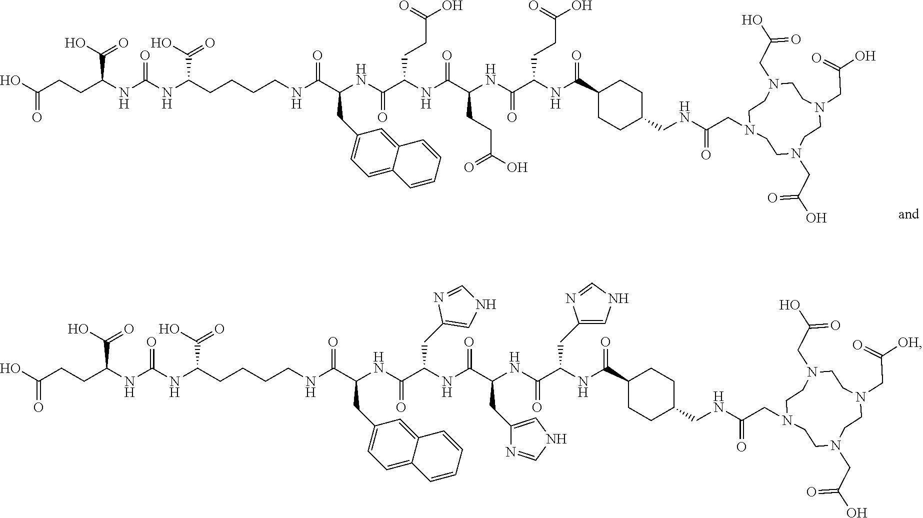 [SMILES: O=C(O)CC[C@H](NC(=O)N[C@@H](CCCCNC(=O)[C@H](Cc1ccc2ccccc2c1)NC(=O)[C@H](CCC(=O)O)NC(=O)[C@H](CCC(=O)O)NC(=O)[C@H](CCC(=O)O)NC(=O)[C@H]1CC[C@H](CNC(=O)CN2CCN(CC(=O)O)CCN(CC(=O)O)CCN(CC(=O)O)CC2)CC1)C(=O)O)C(=O)O ; O=C(O)CC[C@H](NC(=O)N[C@@H](CCCCNC(=O)[C@H](Cc1ccc2ccccc2c1)NC(=O)[C@H](Cc1c[nH]cn1)NC(=O)[C@H](Cc1c[nH]cn1)NC(=O)[C@H](Cc1c[nH]cn1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

even more preferably, the compound has a structure selected from the group consisting of the following structures:

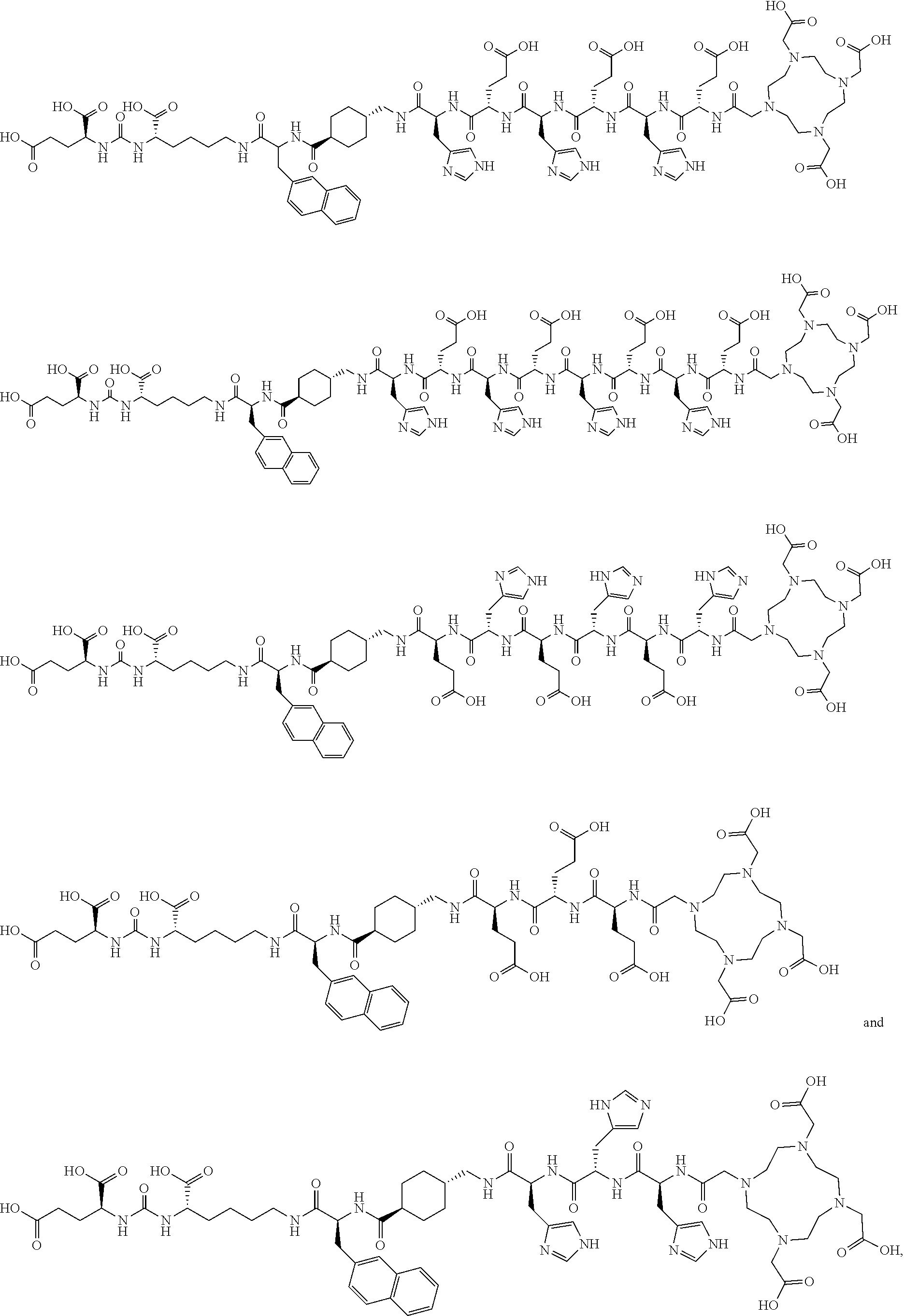

and even more preferably, the compound has a structure selected from the group consisting of the following structures:

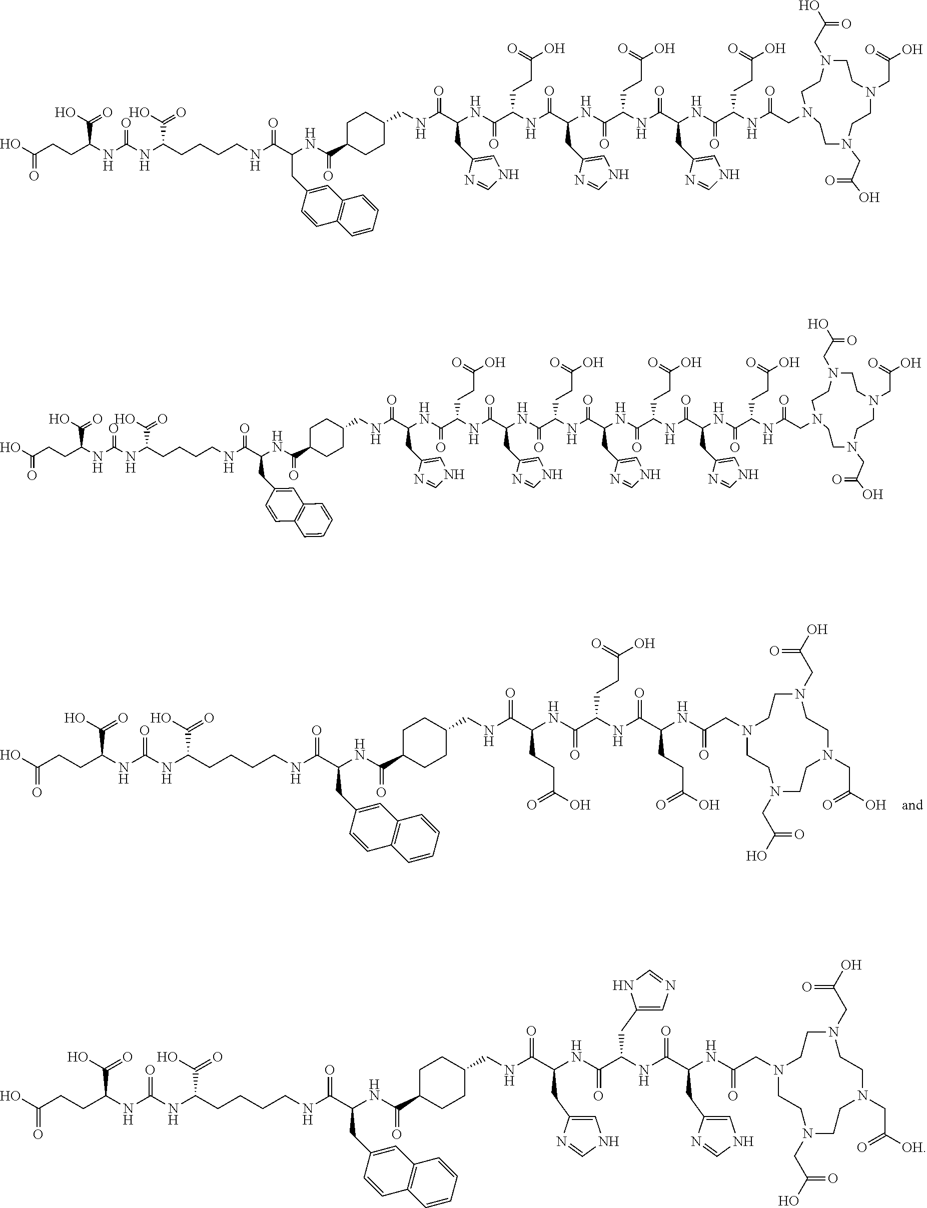

Summarizing the findings of the present invention, the following embodiments are preferred:
1. A compound of formula (1)

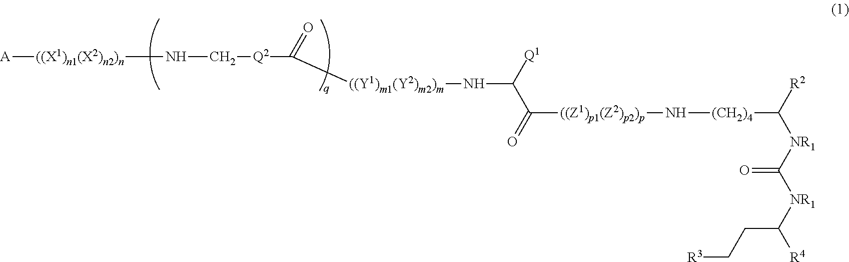

or a pharmaceutically acceptable salt or solvate thereof, wherein R¹ is H or —CH₃, preferably H, wherein R², R³ and R⁴ are independently of each other, selected from the group consisting of —CO₂H, —SO₂H, —SO₃H, —OSO₃H, —PO₂H, —PO₃H and —OPO₃H₂, Q¹ is selected from the group consisting of alkylaryl, arylalkyl, aryl, alkylheteroaryl, heteroarylalkyl and heteroaryl, Q² is selected from the group consisting of aryl, alkylaryl, arylalkyl, cycloalkyl, heterocycloalkyl, heteroaryl, heteroarylalkyl and alkylheteroaryl, A is a chelator residue derived from a chelator selected from the group consisting of 1,4,7,10-tetraazacyclododecane-N,N′,N″,N′″-tetraacetic acid (=DOTA), N,N-bis[2-hydroxy-5-(carboxyethyl)benzyl]ethylenediamine-N,N″-diacetic acid, 1, 4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7, 10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid] (NOPO), 3,6,9, 15-tetraazabicyclo[9.3.1.]pentadeca-1(15),11,13-triene-3,6,9-triacetic acid (=PCTA), N′-{5-[Acetyl(hydroxy)amino]pentyl}-N-[5-(14-[(5-aminopentyl)(hydroxy)amino]-4-oxobutanoyl}amino)pentyl]-N-hydroxysuccinamide (DFO), Diethylenetriaminepentaacetic acid (DTPA), Trans-cyclohexyl-diethylenetriaminepentaacetic acid (CHX-DTPA), 1-oxa-4,7, 10-triazacyclododecane-4,7, 10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl-DTPA (MX-DTPA), X¹, X², Y¹, Y², Z¹ and Z², are independently of each other, charged amino acids, q is an integer of from 0-3, n, m and p, are independently of each other an integer of from 0 to 9, n1, n2, m1, m2, p1, p2, are independently of ach other, an integer of from 0 to 3, and wherein n1+n2>0, m1+m2>0 and p1+p2>0, and wherein n+m+p>0.
2. The compound of embodiment 1, wherein A is a chelator residue having a structure selected from the group consisting of

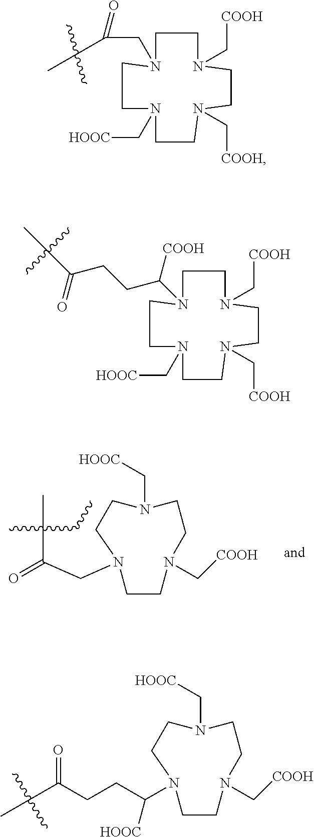

3. The compound of embodiment 1 or 2, wherein (n1+n2)n+(m1+m2)m+(p1+p2)p is at least 2.
4. The compound of anyone of embodiments 1 to 3, wherein (n1+n2)n+(m1+m2)m+(p1+p2)p is an integer of from 2 to 20, preferably of from 2 to 10, more preferably of from 4 to 8, more preferably 6.
5. The compound of any one of embodiments 1 to 4, wherein Q¹ preferably comprises a residue selected from the group consisting of naphtyl, phenyl, biphenyl, indolyl, benzothiazolyl, naphtylmethyl, phenylmethyl, biphenylmethyl, indolylmethyl and benzothiazolylmethyl, more preferably wherein Q¹ is selected from the group consisting of

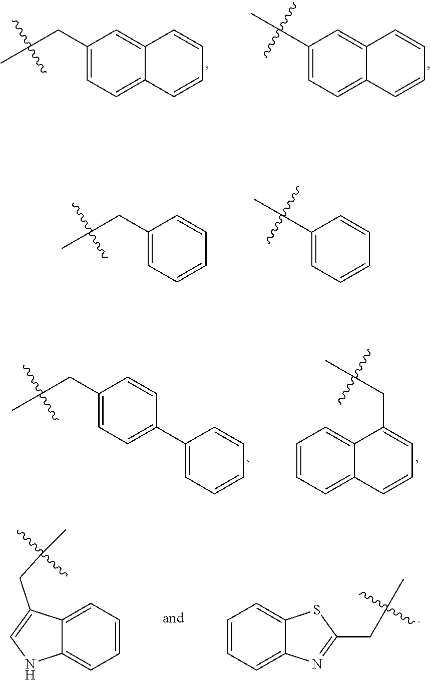

preferably wherein Q¹ is

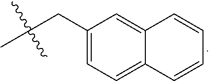 [SMILES: *Cc1ccc2ccccc2c1]

6. The compound of any of one of embodiments 1 to 5, wherein R³, R² and R⁴ are —CO₂H and R¹ is H.
7. The compound of any one of embodiments 1 to 6, wherein Q² is

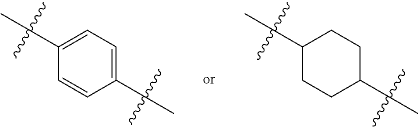 [SMILES: *c1ccc(*)cc1 ; *C1CCC(*)CC1]

preferably

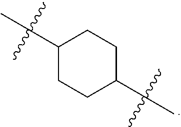 [SMILES: *C1CCC(*)CC1]

8. The compound of any one of embodiments 1 to 7, wherein n1, n2, m1, m2, p1, p2 are, independently of each other, 0 or 1.
9. The compound of any one of embodiments 1 to 8, wherein X¹, X², Y¹, Y², Z¹ and Z² are, independently of each other, at physiological pH, positively or negatively charged amino acids.
10. The compound of embodiment 9, wherein the positively charged amino acids are, independently of each other, selected from the group consisting of arginine, lysine, histidine homoarginine, 3- and 4-substituted arginine analogs, N(delta)-methyl-arginine (deltaMA), canavanine, substituted analogs of canavanine, α-Amino-β-guanidinopropionic acid, γ-guanidinobutyric acid, citrulline, 3-guanidinopropionic acid, 4-{[amino(imino)methyl]amino}butanoic acid, 6-{[amino(imino)methyl]amino}hexanoic acid, 2-Amino-3-guanidinopropionic acid, Arginine hydroxamate, Agmatine (CAS #: 2482-00-0), and NG-Methyl-arginine, preferably, the basic amino acids are, independently of each other, selected from the group consisting of lysine (K), histidine (H) and arginine (R).
11. The compound of embodiment 9 or 10, wherein the negatively charged amino acids are, independently of each other, selected from the group consisting of homoglutamic acid, a sulfonic acid derivative of Cys, cysteic acid, homocysteic acid, aspartic acid (D), glutamic acid (E), preferably, the acidic amino acids are, independently of each other, selected from aspartic acid and glutamic acid.
12. The compound of any one of embodiments 1 to 11, wherein at least one of X¹ and X² is a positively charged amino acid and at least one of X¹ and X² is a negatively charged amino acid.
13. The compound of any one of embodiments 1 to 12, wherein at least one of X¹ and X² is histidine (H) and at least one of X¹ and X² is glutamic acid (E).
14. The compound of any one of embodiments 1 to 13, wherein n1 is 0 or 1 and n2 is 0 or 1.
15. The compound of any one of embodiments 1 to 14, wherein at the building block ((X¹)ₙ₁(X²)ₙ₂)ₙ, has the structure (HE)ₙ or (EH)ₙ, preferably (EH)ₙ.
16. The compound of any one of embodiments 1 to 15, wherein at least one of Y¹ and Y² is a positively charged amino acid and at least one of Y¹ and Y² is a negatively charged amino acid.
17. The compound of any one of embodiments 1 to 16, wherein at least one of Y¹ and Y² is histidine (H) and at least one of Y¹ and Y² is glutamic acid (E).
18. The compound of any one of embodiments 1 to 17, wherein m1 is 0 or 1 and m2 is 0 or 1.
19. The compound of any one of embodiments 1 to 18, wherein at the building block ((Y¹)ₘ₁(Y²)ₘ₂)ₘ, has the structure (HE)ₘ or (EH)ₘ, preferably (EH)ₘ.
20. The compound of any one of embodiments 1 to 19, wherein at least one of Z¹ and Z² is a positively charged amino acid and at least one of Z¹ and Z² is a negatively charged amino acid.
21. The compound of any one of embodiments 1 to 20, wherein at least one of Z¹ and Z² is histidine (H) and at least one of Z¹ and Z² is glutamic acid (E).
22. The compound of any one of embodiments 1 to 21, wherein p1 is 0 or 1 and p2 is 0 or 1.
23. The compound of any one of embodiments 1 to 22, wherein at the building block ((Z¹)ₚ₁(Z²)ₚ₂)ₚ, has the structure (HE)ₚ or (EH)ₚ, preferably (EH)ₚ.
24. The compound of any one of embodiments 1 to 23, wherein n+m+p>1.
25. The compound of any one of embodiments 1 to 24, wherein the building block ((X¹)ₙ₁(X²)ₙ₂)ₙ, has the structure (HE)ₙ or (EH)ₙ and wherein n is of from 2 to 4, more preferably 3, and wherein m is 0 and p is 0.
26. The compound of any one of embodiments 1 to 24, wherein the building block ((Y¹)ₘ₁(Y²)ₘ₂)ₘ, has the structure (HE)ₘ or (EH)ₘ and wherein m is of from 2 to 4, more preferably 3, and wherein n is 0 and p is 0.
27. The compound of any one of embodiments 1 to 24, wherein the building block ((Z¹)ₚ₁(Z²)ₚ₂)ₚ, has the structure (HE)ₚ or (EH)ₚ and wherein p is of from 2 to 4, more preferably 3, and wherein n is 0 and m is 0.
28. The compound of any one of embodiments 1 to 24 having the structure

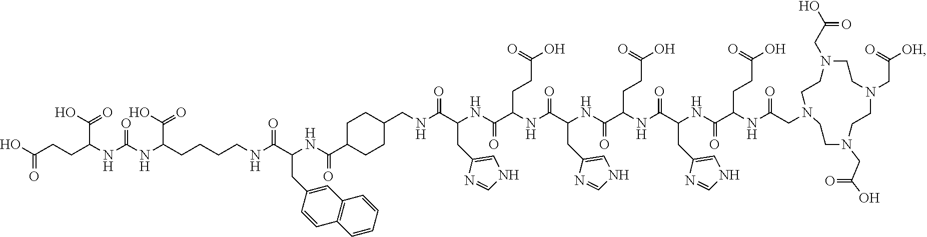

wherein H and E preferably have L configuration.
29. The compound of any one of embodiments 1 to 24 having the structure

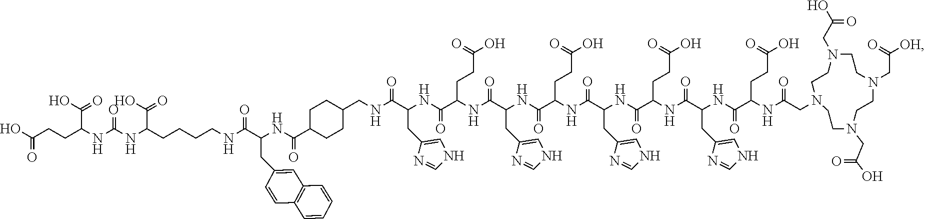

wherein H and E preferably have L configuration.
30. The compound of any one of embodiments 1 to 24 having the structure

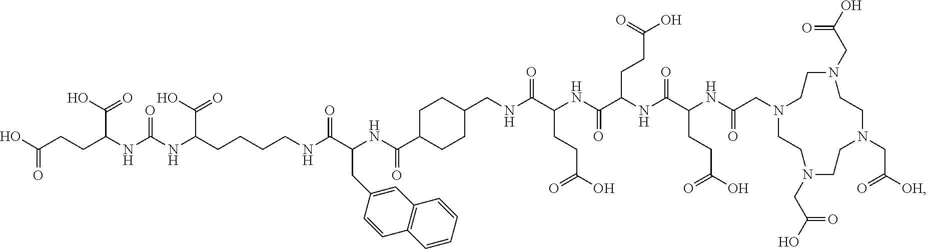

wherein H and E preferably have L configuration.
31. The compound of any one of embodiments 1 to 24 having the structure

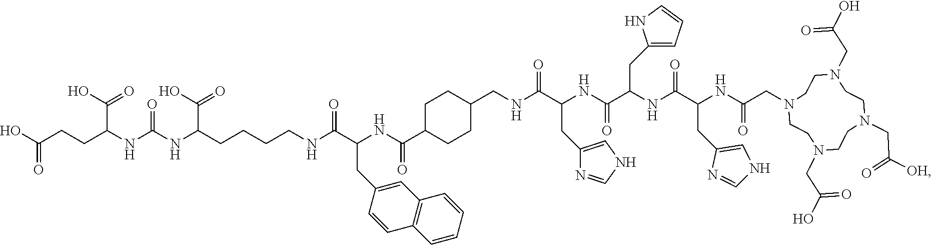

wherein H and E preferably have L configuration.
32. Complex comprising
   (a) a radionuclide, and
   (b) the compound of any one of embodiments 1 to 31 or a pharmaceutically acceptable salt or solvate thereof.
33. The complex of embodiment 32, wherein, the radionuclide is selected from the group consisting ⁸⁹Zr, ⁴⁴Sc, ¹¹¹In, ⁹⁰Y, ⁶⁶Ga, ⁶⁷Ga, ⁶⁸Ga, ¹⁷⁷Lu, ^{99m}Tc, ⁶⁰Cu, ⁶¹Cu, ⁶²Cu, ⁶⁴Cu, ⁶⁶Cu, ⁶⁷Cu, ¹⁴⁹Tb, ¹⁵²Tb, ¹⁵⁵Tb, ¹⁵³Sm, ¹⁶¹Tb, ⁵³Gd, ¹⁵⁵Gd, ¹⁵⁷Gd, ²¹³Bi, ²²⁵Ac, ²³⁰U, ²²³Ra, ¹⁶⁵Er, ⁵²Fe, ⁵⁹Fe, and radionuclides of Pb (such as ²⁰³Pb and ²¹²Pb, ²¹¹Pb, ²¹³Pb, ²¹⁴Pb, ²⁰⁹Pb, ¹⁹⁸Pb, ¹⁹⁷Pb).
34. A pharmaceutical composition comprising a compound of any one of embodiment 1 to 31 or a complex of embodiment 32 or 33.
35. A compound of any one of embodiment 1 to 31 or a complex of embodiment 32 or 33 or a pharmaceutical composition of claim 34 for use in medicine, preferably for treating and/or preventing PSMA expressing cancer, in particular prostate cancer and/or metastases thereof.
36. The compound of embodiment 35, wherein adverse side effects on the salivary glands and/or lacrimal glands are reduced and/or avoided.
37. Compound of any one of embodiment 1 to 31 or a complex of embodiment 32 or 33 or a pharmaceutical composition of claim 34 for use in diagnostics.
38. Compound of embodiment 37 for use in the diagnosis of cancer, preferably of PSMA expressing cancer, in particular of prostate cancer and/or metastases thereof.
39. The compound according to any one of embodiment 35 to 38, wherein the radionuclide is a β-emitter, more preferably ¹⁷⁷Lu and the use is diagnosis, more preferably wherein the activity dosage of the complex is at least 100 kBq/kg body weight, more preferably at least 500 kBq/kg body weight, most preferably at least 1 MBq/kg body weight.
40. The compound according to any one of embodiment 34 to 38, wherein the radionuclide is an α-emitter, more preferably is ²²⁵Ac and the use is therapy, preferably therapy of PSMA expressing cancer, preferably of prostate cancer, wherein the activity dosage of the complex is preferably at least 75 kBq/kg body, more preferably at least 100 kBq/kg body, weight.

All references cited throughout this specification are herewith incorporated by reference with respect to the specifically mentioned disclosure content as well as in their entireties.

### FIGURES

FIG. 1: Pharmacokinetic study with small-animal PET imaging. Time activity curves for kidney after injection of 0.5 nmol ⁶⁸Ga-labeled compounds in LNCaP-tumor-bearing athymic nude mice (right trunk) up to 60 min p.i. SUV=standardized uptake value.

FIGS. 2.1-2.10: Pharmacokinetic study with small-animal PET imaging. Time activity curves for tumor and muscle after injection of 0.5 nmol of the respective ⁶⁸Ga-labeled compound X (see Table 5) in LNCaP-tumor-bearing athymic nude mice (right trunk) up to 60 min p.i. SUV=standardized uptake value.

FIGS. 3.1-3.10: Small-animal PET imaging study. Whole-body maximum intensity projection of 0.5 nmol of the respective ⁶⁸Ga-labeled compound X (see Table 6) in LNCaP-tumor-bearing athymic nude mice (right trunk) 60 min p.i. (FIG. X A) and 120 min p.i. (FIG. X B) obtained from small animal PET imaging.

The following examples shall merely illustrate the invention. Whatsoever, they shall not be construed as limiting the scope of the invention.

### EXAMPLES

All commercially available chemicals were of analytical grade and used without further purification. [⁶⁸Ga]GaCl₄⁻ was obtained from a ⁶⁸Ge/⁶⁸Ga generator (Eckert&Ziegler). [¹⁷⁷Lu]LuCl₃ was obtained from ITG. The compounds were analyzed using reversed-phase high performance liquid chromatography (RP-HPLC; Chromolith RP-18e, 100×4.6 mm; Merck, Darmstadt, Germany). Analytical HPLC runs were performed using a linear gradient 5% (A) (0.1% aqueous TFA) to 50% B (0.1% TFA in CH₃CN)) in 24 min at 1 mL/min.

Analytical HPLC runs were performed using the system Agilent 1200 series (Agilent Technologies, Santa Clara, Calif., USA). UV absorbance was measured at 220 and 280 nm, respectively. For mass spectrometry a LC-MS SQ300 (Perkin Elmer, Waltham, Mass., USA) was used.

The precursors PSMA-617 (2-[3-(1-Carboxy-5-{3-naphthalen-2-yl-2-[(4-{[2-(4,7,10-tris-carboxymethyl-1,4,7,10-tetraaza-cyclododec-1-yl)-acetylamino]-methyl}-cyclohexanecarbonyl)-amino]-propionylamino}-pentyl)-ureido]-pentanedioic acid) and PSMA-10 ([Glu-urea-Lys(Ahx)]₂-HBED-CC) were purchased from ABX, Radeberg, Germany.

### I. Synthesis

### I.1 Synthesis of Compounds PS1-PS10

Unless indicated otherwise, the compounds have been synthesized as follows:

The synthesis of the pharmacophore Glu-urea-Lys was performed according to Schäfer M et al. (2012), EJNMMI Res. 2(1):23. Briefly, the synthesis started with the formation of the isocyanate of the glutamyl moiety using triphosgene. A resin-immobilized (2-chloro-tritylresin, Merck, Darmstadt) ε-allyloxycarbonyl protected lysine was added and reacted for 16 h with gentle agitation. The resin was filtered off and the allyloxy-protecting group was removed by reacting twice with Pd(PPh₃)₄ (0.3 eq.) and morpholine (15 eq.) under ambient conditions (1 h, RT). The resin was split and the linkers were introduced by standard Fmoc solid phase protocols. According to the amino acid sequence of PS1-PS10 the Fmoc-protected amino acids (4 eq. each) with HATU (4 eq.) and DIPEA (10 eq.) were coupled in DMF. After coupling the last amino acid of the sequence, tris(tBu)DOTA (tris(tBu)-ester of 1,4,7,10tetraazacyclododecan-1,4,7,10-tetraacetic acid) (4 eq. each) with HATU (4 eq.) and DIPEA (10 eq.) was coupled in DMF. The product was cleaved from the resin for 3 hours at RT using TFA/TIPS/H2O (95/2.5/2.5, v/v/v).

All products were purified using RP-HPLC and identified with mass spectrometry.

Purification was done using a semipreparative column (SemiPrep, Chromolith RP-18e, 100×10 mm; Merck, Darmstadt, Germany). Solvent A consisted of 0.1% aqueous TFA and solvent B was 0.1% TFA in CH₃CN.

The following compounds were synthesized:

TABLE A
Synthesized Compounds

| Compound X | Structure (PSMA = “Lys-urea-Glu”) |
|---|---|
| PS-1 | DOTA-CHx-2NaI-(EH)3-PSMA |
| PS-2 | DOTA-(EH)3-CHx-2NaI-PSMA |
| PS-3 | DOTA-Chx-(EH)3-2NaI-PSMA |
| PS-4 | DOTA-(EH)4-CHx-2NaI-PSMA |
| PS-4.2 | DOTA-(HE)3-CHx-2NaI-PSMA |
| PS-5 | DOTA-(E)3-CHx-2NaI-PSMA |
| PS-6 | DOTA-CHx-2NaI-(E)3-PSMA |
| PS-7 | DOTA-(H)3-CHx-2NaI-PSMA |
| PS-8 | DOTA-CHx-2NaI-(H)3-PSMA |
| PS-9 | DOTA-CHx-(E)3-2NaI-PSMA |
| PS10 | DOTA-CHx-(H)3-2NaI-PSMA |

### I.2 ⁶⁸Ga—Labeling

The precursor peptides [2 nmol in HEPES buffer (1 M, pH 7), 40 μL] were added to 40 μL [⁶⁸Ga]GaCl₄⁻ (˜30 MBq). The reaction mixture was incubated at 95° C. for 15 minutes. The radiochemical yield (RCY) was determined by HPLC.

### I.3 ¹⁷⁷Lu—Labeling

The precursor peptides [1 nmol in HEPES buffer (0.1 M, pH 7.2), 50 μL] were added to 10 μL [¹⁷⁷Lu]LuCl₃ (˜30 MBq). The reaction mixture was incubated at 95° C. for 15 minutes. The radiochemical yield (RCY) was determined by HPLC.

### I.4 Example 1: Synthesis of DOTA-(EH)₃—CHx-2NaI-Lys-urea-Glu and DOTA-CHx-2NaI-(EH)₃-Lys-urea-Glu

The synthesis of the pharmacophore Glu-urea-Lys was performed according to Schäfer M et al. (2012), EJNMMI Res. 2(1):23. Briefly, the synthesis started with the formation of the isocyanate of the glutamyl moiety using triphosgene. A resin-immobilized (2-chloro-tritylresin, Merck, Darmstadt) F-allyloxycarbonyl protected lysine was added and reacted for 16 h with gentle agitation. The resin was filtered off and the allyloxy-protecting group was removed by reacting twice with Pd(PPh₃)₄(0.3 eq.) and morpholine (15 eq.) under ambient conditions (1 h, RT). The resin was split and the linkers were introduced by standard Fmoc solid phase protocols.

Synthesis of DOTA-(EH)₃—CHx-2NaI-Lys-urea-Glu

In a first step Fmoc-2-NaI—OH and N-Fmoc-tranexamic acid (4 eq. each) with HATU (4 eq.) and DIPEA (10 eq.) were coupled in DMF. For the introduction of (HE)₃ the coupling of Fmoc-His(Trt)-OH and Fmoc-Glu(otBu)-OH (4 eq.) was performed using HATU (4 eq.) and DIPEA (10 eq.) in DMF. In order to form (HE)₃ the coupling of Fmoc-His(Trt)-OH and Fmoc-Glu(otBu)-OH was repeated, respectively. Subsequently, tris(tBu)DOTA (tris(tBu)-ester of 1,4,7,10tetraazacyclododecan-1,4,7,10-tetraacetic acid) (4 eq. each) with HATU (4 eq.) and DIPEA (10 eq.) was coupled in DMF. The product was cleaved from the resin for 3 hours at RT using TFA/TIPS/H2O (95/2.5/2.5, v/v/v).

Synthesis of DOTA-CHx-2NaI-(EH)₃-Lys-urea-Glu

In a first step coupling of Fmoc-His(Trt)-OH and Fmoc-Glu(otBu)-OH (4 eq.) was performed using HATU (4 eq.) and DIPEA (10 eq.) in DMF. In order to form (HE)₃ the coupling of Fmoc-His(Trt)-OH and Fmoc-Glu(otBu)-OH was repeated, respectively. Afterwards Fmoc-2-NaI—OH and N-Fmoc-tranexamic acid (4 eq. each) with HATU (4 eq.) and DIPEA (10 eq.) were coupled in DMF. Subsequently, tris(tBu)DOTA (tris(tBu)-ester of 1,4,7,10tetraazacyclododecan-1,4,7,10-tetraacetic acid) (4 eq. each) with HATU (4 eq.) and DIPEA (10 eq.) was coupled in DMF. The product was cleaved from the resin for 3 hours at RT using TFA/TIPS/H2O (95/2.5/2.5, v/v/v).

All products were purified using RP-HPLC and identified with mass spectrometry.

Purification was done using a NUCLEOSIL column (VP250/21, 5 μm particles, 120-5 C18; Macherey-Nagel, Duren, Germany). Solvent A consisted of 0.1% aqueous TFA and solvent B was 0.1% TFA in CH3CN.

177Lu—Labeling

The precursor peptides [1 nmol in HEPES buffer (0.1 M, pH 7.2), 50 μL] were added to 10 μL [¹⁷⁷Lu]LuCl₃ (˜30 MBq). The reaction mixture was incubated at 95° C. for 30 minutes. The radiochemical yield (RCY) was determined by HPLC.

### II. Cell Assays

### II.1 Cell Culture

PSMA⁺ LNCaP cells (ATCC CRL-1740) were cultured in RPMI medium. Cells were grown at 37° C. in humidified air with 5% C02 and were harvested using trypsin-ethylenediaminetetraacetic acid.

### II.2 Cell Binding and Internalization

The competitive cell binding assay and internalization experiments were performed according to Eder M et al. (2012), Bioconjug Chem 23(4):688.

Briefly, the cells (10⁵ per well) were incubated with a 0.8 nM solution of ⁶⁸Ga-labeled radioligand [Glu-urea-Lys(Ahx)]₂-HBED-CC (PSMA-10, precursor ordered from ABX, Radeberg, Germany) in the presence of 12 different concentrations of analyte (0-5000 nM, 100 μL/well). After incubation, the mixture was removed and the wells were washed 3 times with PBS using a multiscreen vacuum manifold (Millipore, Billerica, Mass.). Cell-bound radioactivity was measured using a gamma counter (Packard Cobra II, GMI, Minnesota, USA). The 50% inhibitory concentration (IC50) values were calculated by fitting the data using a nonlinear regression algorithm (GraphPad Software).

For internalization experiments, 10⁵ cells per well were seeded in poly-L-lysine coated 24-well cell culture plates 24 h before incubation. After washing, the cells were incubated with 30 nM of the ⁶⁸Ga- or ¹⁷⁷Lu-radiolabeled compound for 45 min at 37° C. and at 4° C., respectively. Cellular uptake was terminated by washing 3 times with 1 mL of ice-cold PBS. To remove surface-bound radioactivity, cells were incubated twice with 0.5 mL glycine-HCl in PBS (50 mM, pH=2.8) for 5 min. The cells were washed with 1 mL of ice-cold PBS and lysed using 0.3 N NaOH (0.5 mL). The surface-bound and the internalized fractions were measured in a gamma counter. The cell uptake was calculated as percent of the initially added radioactivity bound to 10⁵ cells [% ID/10⁵ cells].

Statistical Aspects

All experiments were performed at least in triplicate and repeated at least for three times.

Quantitative data were expressed as mean±SD. If applicable, means were compared using Student's t test. P values<0.05 were considered statistically significant.

### III. μPET Imaging

For μPET imaging, mice were anaesthetized (2% isoflurane), placed into a small animal PET scanner (PET Focus, Siemens) and injected with 500 pmol ⁶⁸Ga-labeled peptide per mouse. A 60 min dynamic scan and static scans at 60 and 120 min p.i. were performed. Images were reconstructed and converted to standardized uptake value (SUV) shown in maximum intensity projection (MIP) images and time activity curves. Quantitation was done using a ROI (region of interest) technique and expressed as SUVmean.

### IV. Results

The internalization efficiency of the compounds was determined in order to investigate the influence of the linkers on the binding properties. The results are summarized in Table 1. Both synthesized compounds show significantly higher specific cell surface binding and specific internalization as compared to the reference PSMA-617.

TABLE 1
Internalization data of the investigated compounds Glu-urea-
Lys-2-Nal-Chx-(HE)₃-DOTA and Glu-urea-Eys-(HE)₃-2-Nal-Chx-DOTA
compared to the reference PSMA-617 labeled with¹⁷⁷ Lu.

| | Specific cell | Specific |
|---|---|---|
| | surface bound | internalized |
| | [% ID/10⁵ cells] | [% ID/10⁵ cells] |
| DOTA-CHx-NaI-Lys-urea-Glu | 4.36 ± 1.33 | 2.94 ± 0.35 |
| (PSMA-617)) | | |
| DOTA-(EH)₃-CHx-2NaI-Lys- | 9.55 ± 1.61 | 6.00 ± 0.71 |
| urea-Glu | | |
| DOTA-CHx-2NaI-(EH)₃-Lys- | 6.84 ± 0.06 | 4.32 ± 0.03 |
| urea-Glu | | |

TABLE 2
Specific Binding Affinity

| | IC₅₀ (nM) |
|---|---|
| PSMA-617 | 21.77 ± 3.13 |
| PS-1 | 55.98 ± 7.70 |
| PS-2 | 36.96 ± 11.36 |
| PS-3 | 43.13 ± 9.63 |
| PS-4 | 46.68 ± 37.30 |
| PS-4.2 | 27.86 ± 6.93 |
| PS-5 | 10.40 ± 2.94 |
| PS-6 | 28.55 ± 8.42 |
| PS-7 | 22.68 ± 6.47 |
| PS-8 | 78.64 ± 44.14 |
| PS-9 | 23.37 ± 9.70 |
| PS10 | 53.98 ± 20.89 |

TABLE 3
Cell surface binding and internalization
of the ⁶⁸Ga-labeled compounds.

| | Cell Surface binding | Internalization |
|---|---|---|
| | [% ID/10⁵ cells] | [% ID/10⁵ cells] |
| PSMA-617 | 0.792 ± 0.134 | 0.504 ± 0.051 |
| blocked | 0.063 ± 0.014 | 0.117 ± 0.032 |
| PS-1 | 1.162 ± 0.183 | 0.676 ± 0.155 |
| blocked | 0.069 ± 0.013 | 0.212 ± 0.037 |
| PS-2 | 1.333 ± 0.127 | 0.797 ± 0.122 |
| blocked | 0.060 ± 0.017 | 0.187 ± 0.032 |
| PS-3 | 1.023 ± 0.140 | 0.557 ± 0.042 |
| blocked | 0.183 ± 0.025 | 0.317 ± 0.025 |
| PS-4 | 1.420 ± 0.113 | 0.676 ± 0.070 |
| blocked | 0.083 ± 0.029 | 0.151 ± 0.048 |
| PS-4.2 | 1.437 ± 0.138 | 0.676 ± 0.070 |
| blocked | 0.080 ± 0.010 | 0.113 ± 0.023 |
| PS-5 | 1.003 ± 0.127 | 0.725 ± 0.269 |
| blocked | 0.055 ± 0.013 | 0.095 ± 0.026 |
| PS-6 | 0.767 ± 0.156 | 0.607 ± 0.136 |
| blocked | 0.050 ± 0.020 | 0.156 ± 0.023 |
| PS-7 | 0.817 ± 0.047 | 0.673 ± 0.093 |
| blocked | 0.123 ± 0.015 | 0.207 ± 0.021 |
| PS-8 | 0.945 ± 0.030 | 0.600 ± 0.066 |
| blocked | 0.038 ± 0.010 | 0.093 ± 0.033 |
| PS-9 | 0.650 ± 0.083 | 0.410 ± 0.090 |
| blocked | 0.060 ± 0.021 | 0.138 ± 0.046 |
| PS10 | 0.580 ± 0.046 | 0.523 ± 0.078 |
| blocked | 0.027 ± 0.006 | 0.083 ± 0.006 |

TABLE 4
Specific cell surface binding and internalization
of the ⁶⁸Ga-labeled compounds.

| | Specific Cell Surface | |
|---|---|---|
| ⁶⁸Ga-labeled | Binding | Specific Internalization |
| compound X | [% ID/10⁵ cells] | [% ID/10⁵ cells] |
| PSMA-617 | 0.72 ± 0.06 | 0.39 ± 0.06 |
| PS-1 | 1.09 ± 0.17 | 0.46 ± 0.11 |
| PS-2 | 1.27 ± 0.15 | 0.61 ± 0.15 |
| PS-3 | 0.84 ± 0.03 | 0.24 ± 0.02 |
| PS-4 | 1.33 ± 0.09 | 0.52 ± 0.04 |
| PS-4.2 | 1.35 ± 0.28 | 0.71 ± 0.28 |
| PS-5 | 0.94 ± 0.13 | 0.63 ± 0.28 |
| PS-6 | 0.71 ± 0.12 | 0.45 ± 0.12 |
| PS-7 | 0.69 ± 0.11 | 0.47 ± 0.11 |
| PS-8 | 0.90 ± 0.03 | 0.51 ± 0.05 |
| PS-9 | 0.59 ± 0.09 | 0.27 ± 0.07 |
| PS10 | 0.55 ± 0.08 | 0.44 ± 0.08 |

Pharmacokinetic Study in PSMA+ Tumor Bearing Balb/c Nude Mice:

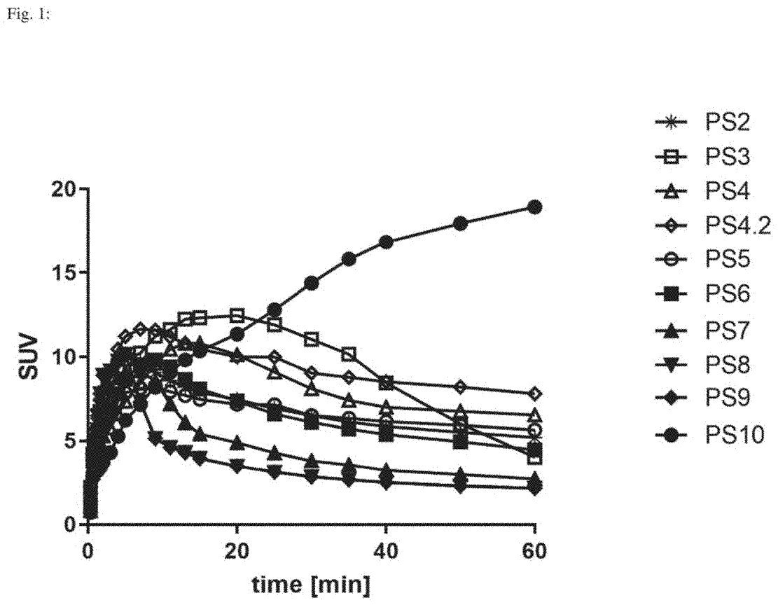

The time activity curves for kidney after injection of 0.5 nmol ⁶⁸Ga-labeled compounds in LNCaP-tumor-bearing athymic nude mice (right trunk) up to 60 min p.i. are shown in FIG. 1. (SUV=standardized uptake value)

Further, the time activity curves for tumor and muscle after injection of 0.5 nmol ⁶⁸Ga-labeled compound X in LNCaP-tumor-bearing athymic nude mice (right trunk) up to 60 min p.i. (SUV=standardized uptake value) is shown in the following figures:

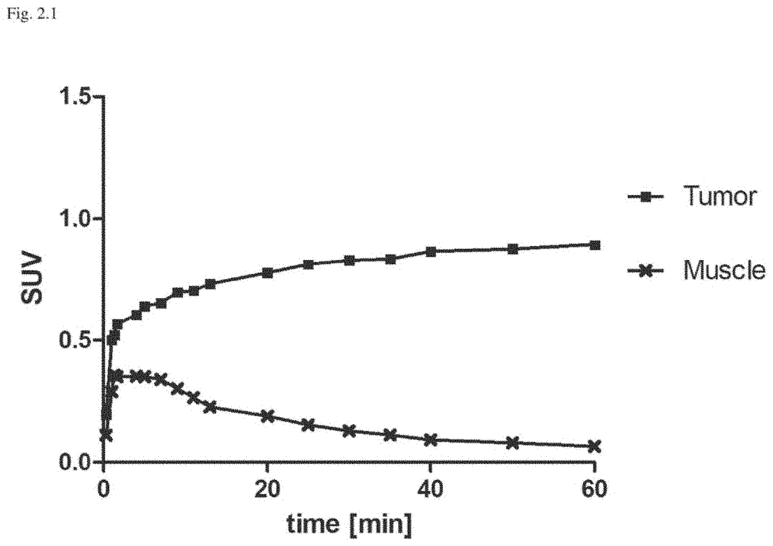

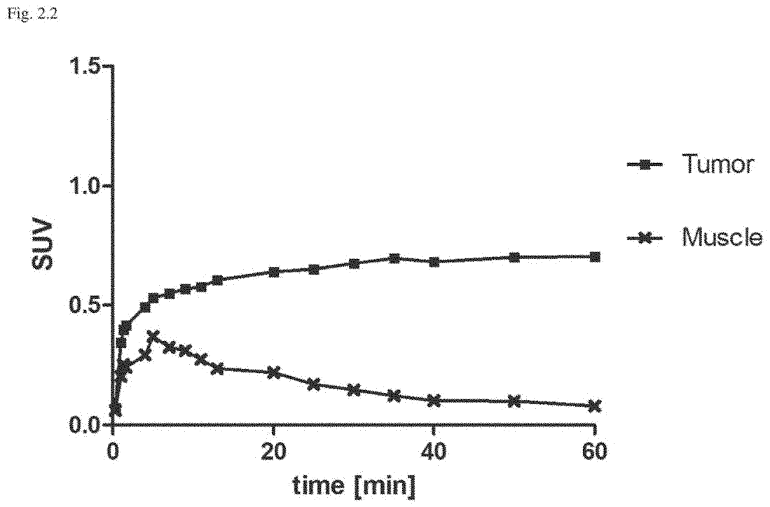

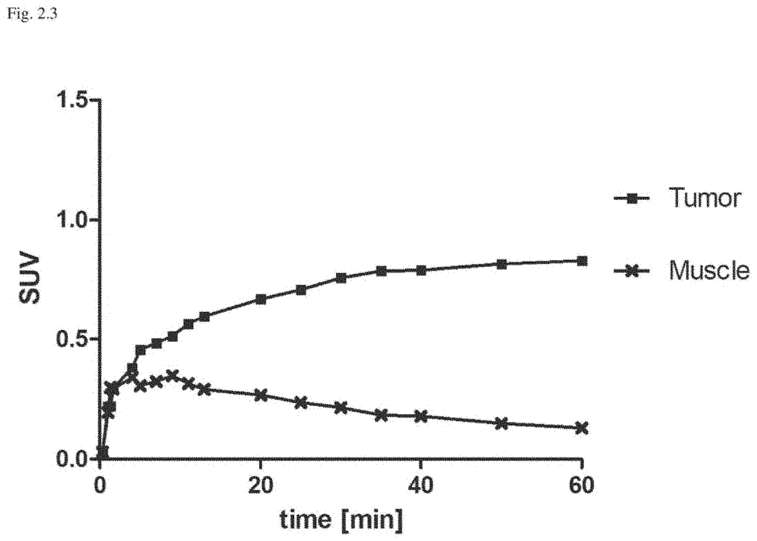

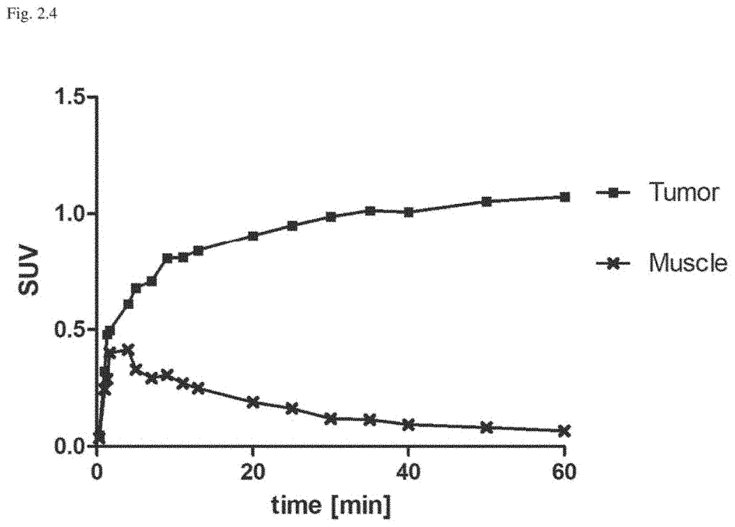

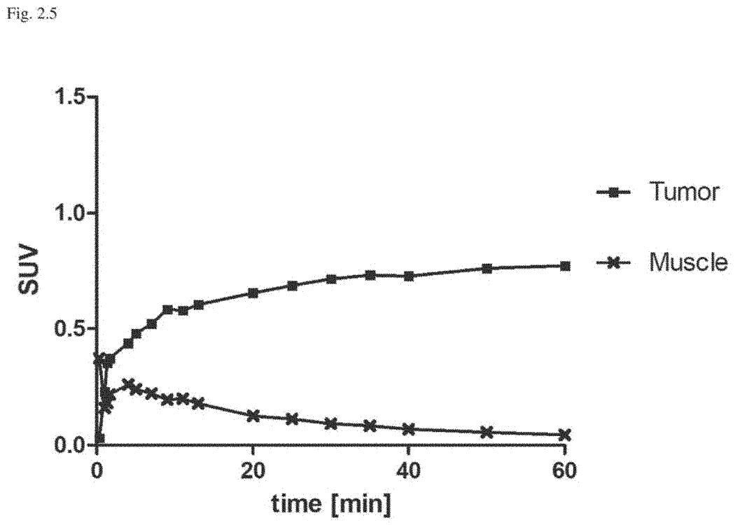

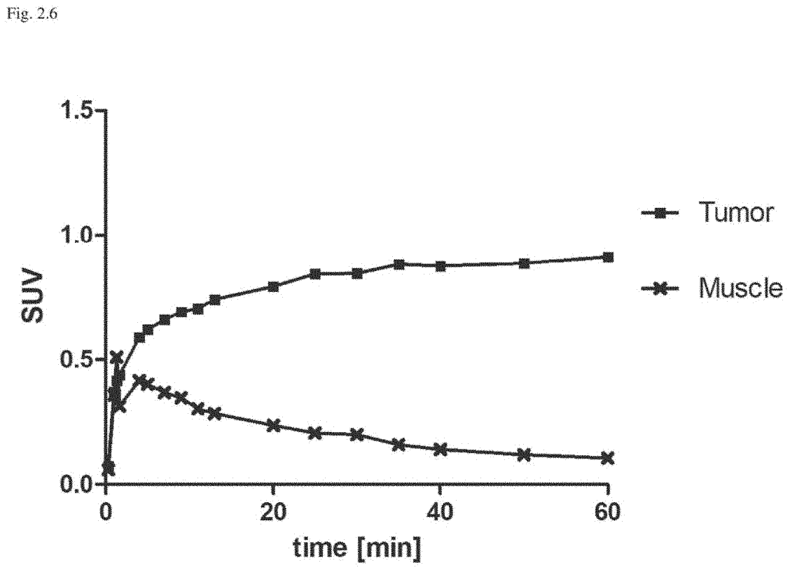

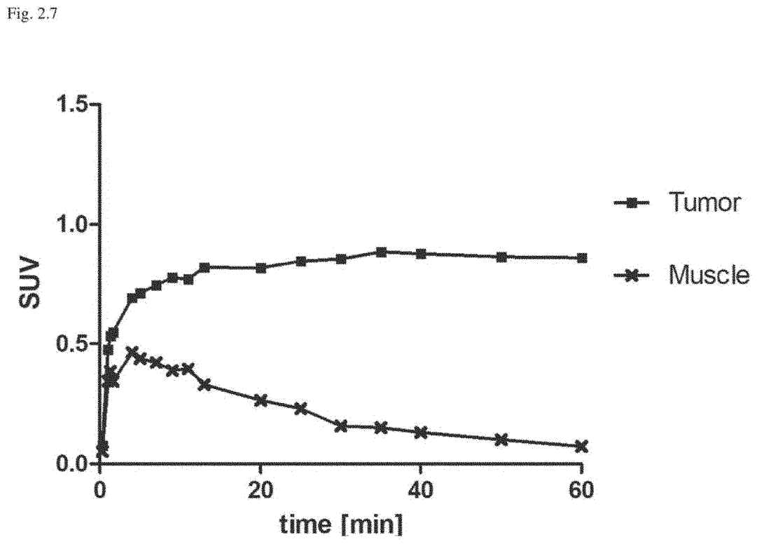

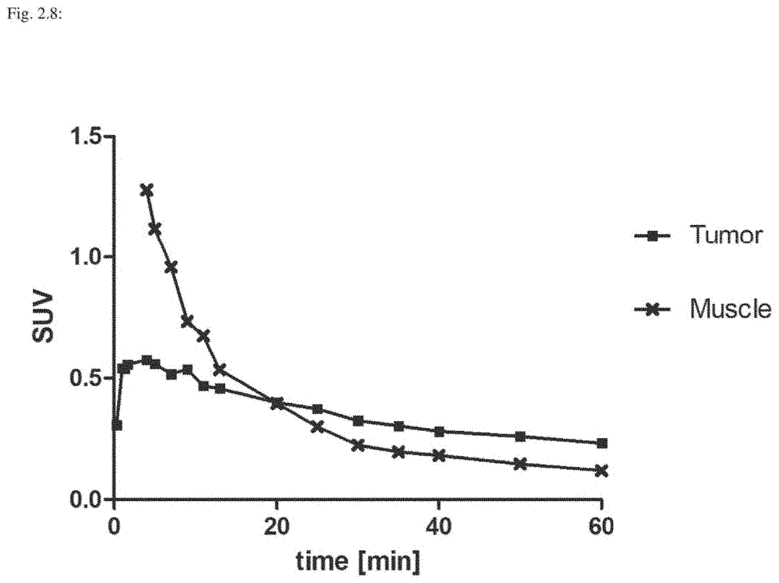

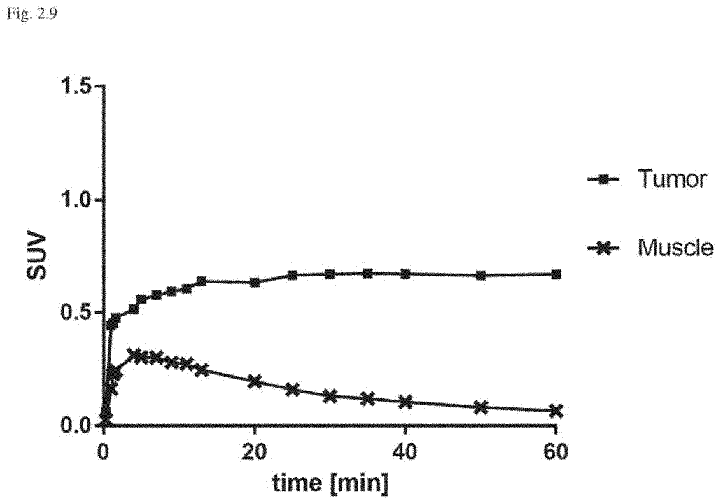

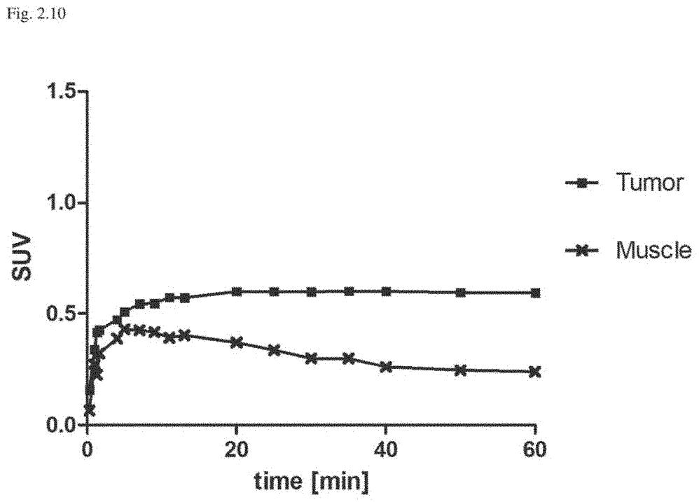

TABLE 5

| ⁶⁸Ga-labeled | Time activity curve |
|---|---|
| compound X | Tumor/Muscle |
| PS-2 | FIG. 2.1 |
| PS-3 | FIG. 2.2 |
| PS-4 | FIG. 2.3 |
| PS-4.2 | FIG. 2.4 |
| PS-5 | FIG. 2.5 |
| PS-6 | FIG. 2.6 |
| PS-7 | FIG. 2.7 |
| PS-8 | FIG. 2.8 |
| PS-9 | FIG. 2.9 |
| PS10 | FIG. 2.10 |

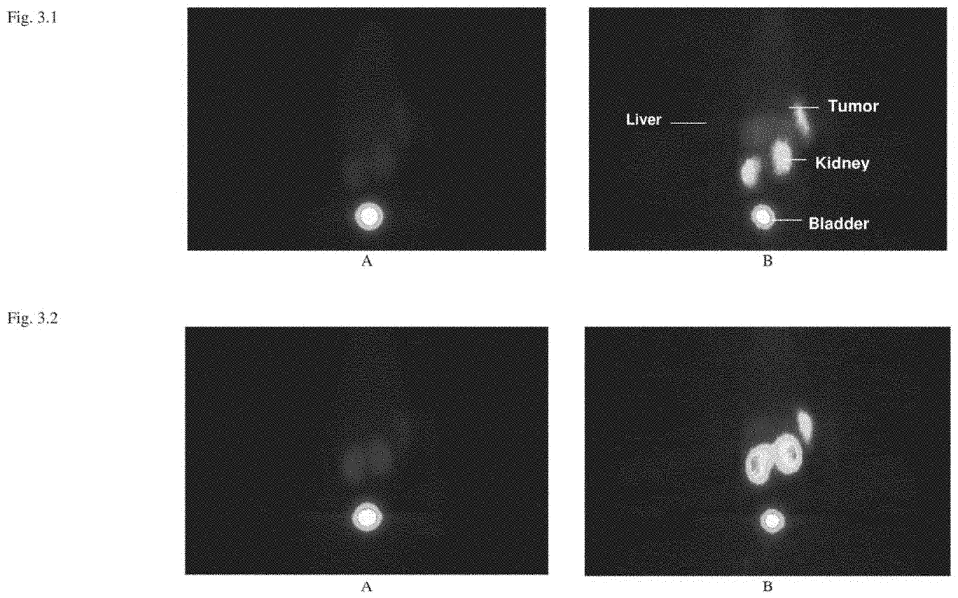

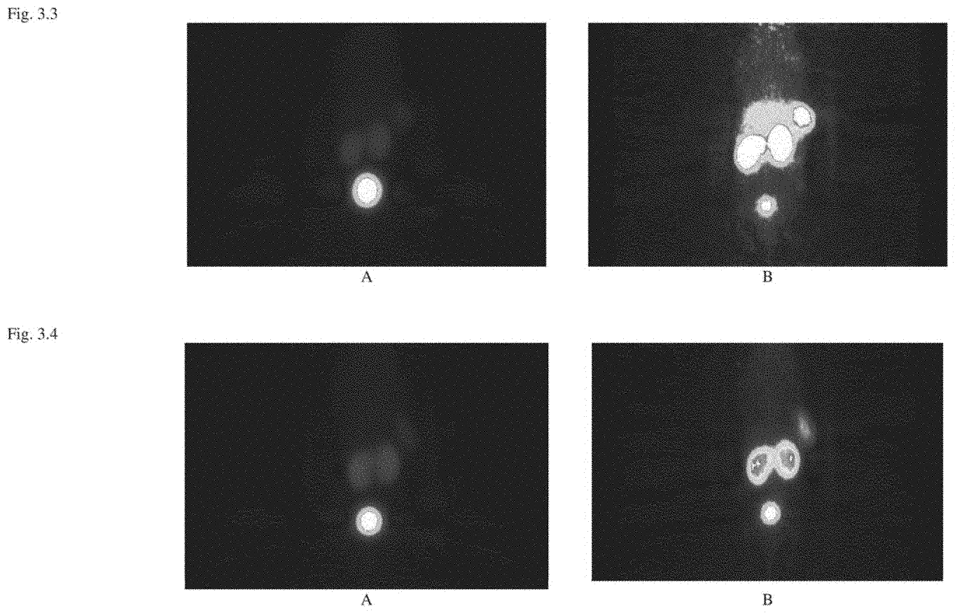

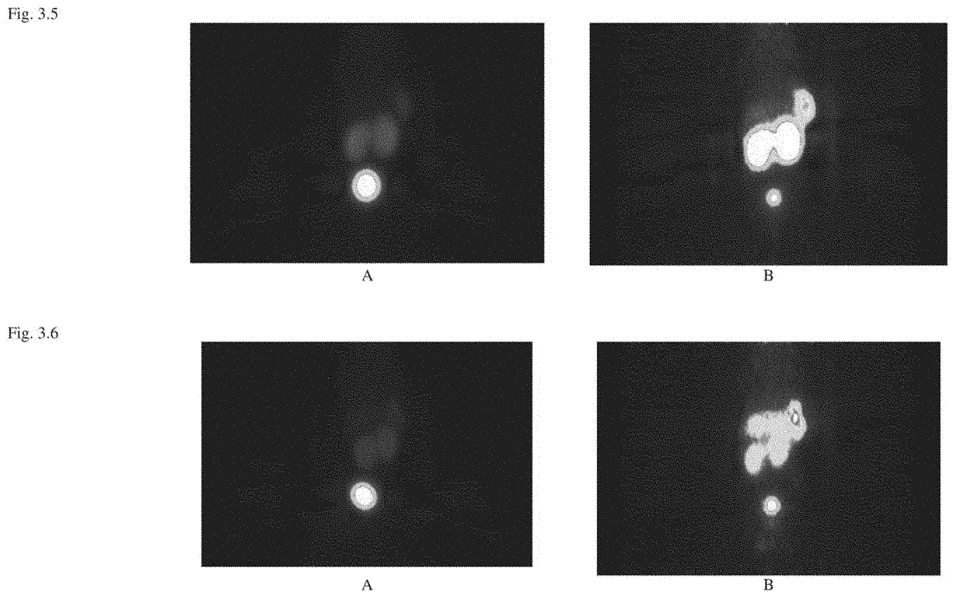

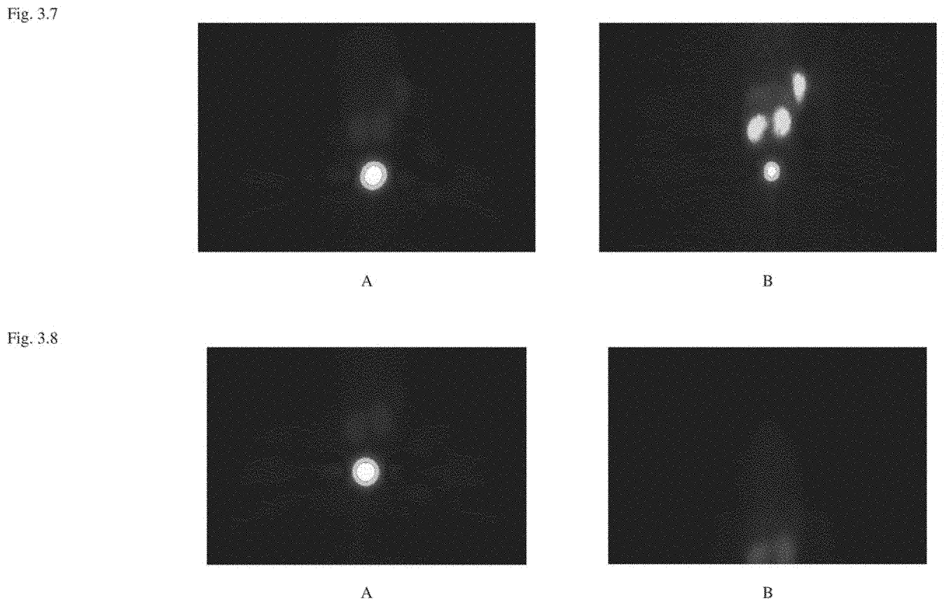

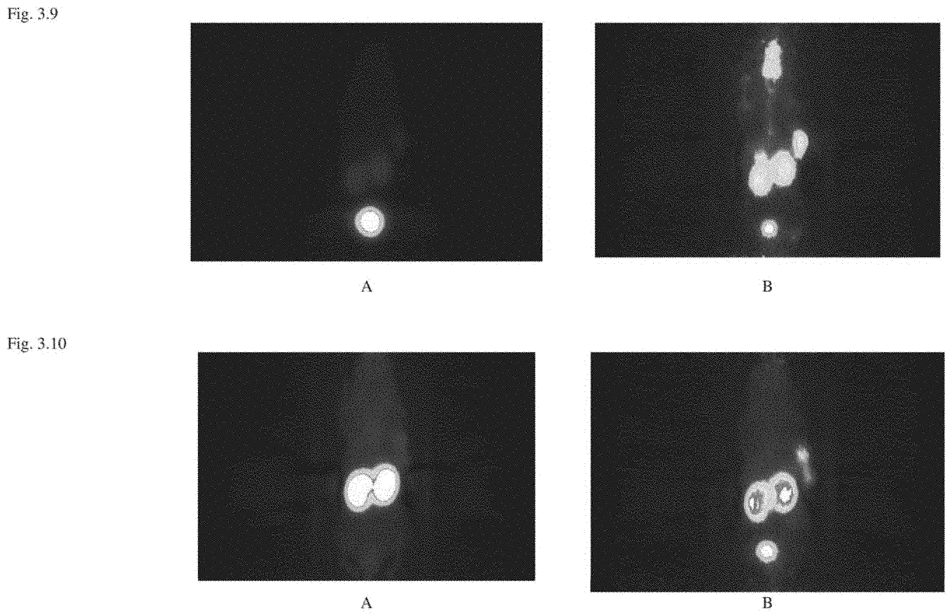

TABLE 6.1-6-10
Small-animal PET imaging study. Whole-body maximum intensity
projection of 0.5 nmol ⁶⁸Ga-labeled compound X in LNCaP-
tumor-bearing athymic nude mice (right trunk) 60 and after
120 min p.i. obtained from small animal PET imaging.

| ⁶⁸Ga-labeled | | |
|---|---|---|
| compound X | 1 h p.i. | 2 h p.i. |
| PS-1 | FIG. 3.1 A | FIG. 3.1 B |
| PS-2 | FIG. 3.2 A | FIG. 3.2 B |
| PS-3 | FIG. 3.3 A | FIG. 3.3 B |
| PS-4 | FIG. 3.4 A | FIG. 3.4 B |
| PS-4.2 | FIG. 3.5 A | FIG. 3.5 B |
| PS-6 | FIG. 3.6 A | FIG. 3.6 B |
| PS-7 | FIG. 3.7 A | FIG. 3.7 B |
| PS-8 | FIG. 3.8 A | FIG. 3.8 B |
| PS-9 | FIG. 3.9 A | FIG. 3.9 B |
| PS10 | FIG. 3.10 A | FIG. 3.10 B |

1. A compound of formula (1) 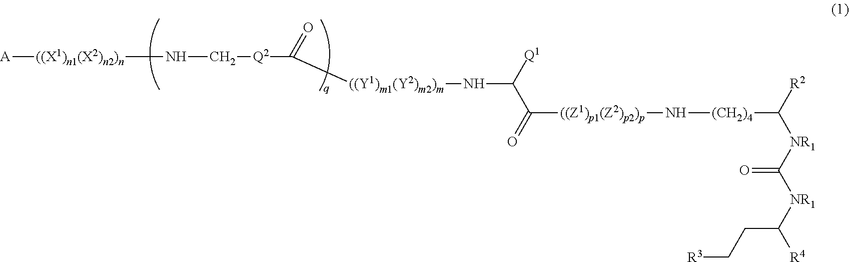
or a pharmaceutically acceptable salt or solvate thereof,
wherein R¹ is H or —CH₃, preferably H,
wherein R², R³ and R⁴ are independently of each other, selected from the group consisting of —CO₂H, —SO₂H, —SO₃H, —OSO₃H, —PO₂H, —PO₃H and —OPO₃H₂,
Q¹ is selected from the group consisting of alkylaryl, arylalkyl, aryl, alkylheteroaryl, heteroarylalkyl and heteroaryl,
Q² is selected from the group consisting of aryl, alkylaryl, arylalkyl, cycloalkyl, heterocycloalkyl, heteroaryl, heteroarylalkyl and alkylheteroaryl,
A is a chelator residue derived from a chelator selected from the group consisting of 1,4,7,10-tetraazacyclododecane-N,N′,N″,N′″-tetraacetic acid (=DOTA), N,N″-bis[2-hydroxy-5-(carboxyethyl)benzyl]ethylenediamine-N,N″-diacetic acid, 1,4,7-triazacyclononane-1,4,7-triacetic acid (=NOTA), 2-(4,7-bis(carboxymethyl)-1,4,7-triazonan-1-yl)pentanedioic acid, (NODAGA), 2-(4,7, 10-tris(carboxymethyl)-1,4,7,10-tetraazacyclododecan-1-yl)pentanedioic acid (DOTAGA), 1,4,7-riazacyclononane phosphinic acid (TRAP), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid] (NOPO), 3,6,9, 15-tetraazabicyclo[9.3.1.]pentadeca-1(15),11,13-triene-3,6,9-triacetic acid (=PCTA), N′-{5-[Acetyl(hydroxy)amino]pentyl}-N-[5-({4-[(5-aminopentyl)(hydroxy)amino]-4-oxobutanoyl}amino)pentyl]-N-hydroxysuccinamide (DFO), Diethylenetriaminepentaacetic acid (DTPA), Trans-cyclohexyl-diethylenetriaminepentaacetic acid (CHX-DTPA), 1-oxa-4,7, 10-triazacyclododecane-4,7, 10-triacetic acid (oxo-Do3A) p-isothiocyanatobenzyl-DTPA (SCN-Bz-DTPA), 1-(p-isothiocyanatobenzyl)-3-methyl-DTPA (1 B3M), 2-(p-isothiocyanatobenzyl)-4-methyl-DTPA (1 M3B) and 1-(2)-methyl-4-isocyanatobenzyl-DTPA (MX-DTPA),
X¹, X², Y¹, Y², Z¹ and Z², are independently of each other, charged amino acids,
q is an integer of from 0-3,
n, m and p, are independently of each other an integer of from 0 to 9,
n1, n2, m1, m2, p1, p2, are independently of ach other, an integer of from 0 to 3,
and wherein n1+n2>0, m1+m2>0 and p1+p2>0,
and wherein n+m+p>0.

2. The compound of claim 1, wherein A is a chelator residue having a structure selected from the group consisting of

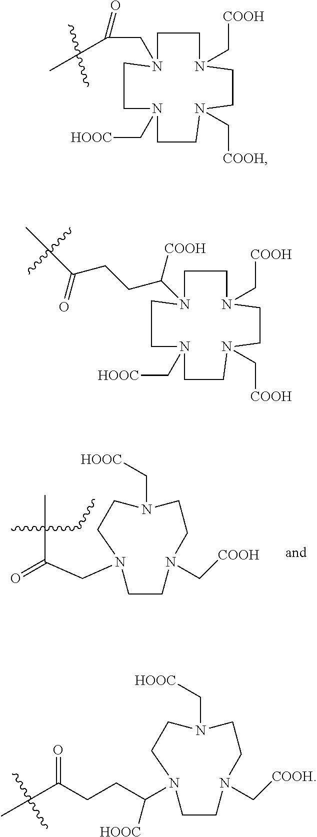

3. The compound of claim 1, wherein (n1+n2)n+(m1+m2)m+(p1+p2)p is at least 2.

4. The compound of claim 1, wherein (n1+n2)n+(m1+m2)m+(p1+p2)p is an integer of from 2 to 20, preferably of from 2 to 10, more preferably of from 4 to 8, more preferably 6.

5. The compound of claim 1, wherein Q¹ preferably comprises a residue selected from the group consisting of naphtyl, phenyl, biphenyl, indolyl, benzothiazolyl, naphtylmethyl, phenylmethyl, biphenylmethyl, indolylmethyl and benzothiazolylmethyl, more preferably wherein Q¹ is selected from the group consisting of 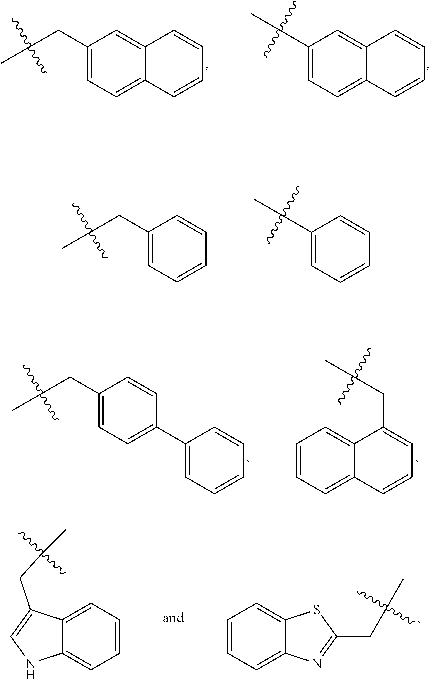
preferably wherein Q¹ is

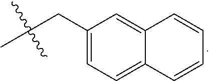 [SMILES: *Cc1ccc2ccccc2c1]

6. The compound of claim 1, wherein R³, R² and R⁴ are —CO₂H and R¹ is H.

7. The compound of claim 1, wherein Q² is 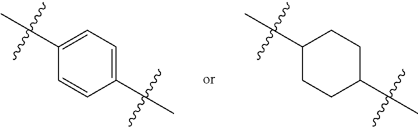 [SMILES: *c1ccc(*)cc1 ; *C1CCC(*)CC1]
preferably

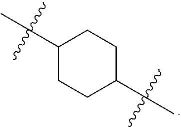 [SMILES: *C1CCC(*)CC1]

8. The compound of claim 1, wherein X¹, X², Y¹, Y², Z¹ and Z², are independently of each other, at physiological pH, positively or negatively charged amino acids, and wherein the positively charged amino acids are, independently of each other, selected from the group consisting of arginine, lysine, histidine homoarginine, 3- and 4-substituted arginine analogs, N(delta)-methyl-arginine (deltaMA), canavanine, substituted analogs of canavanine, α-Amino-β-guanidinopropionic acid, γ-guanidinobutyric acid, citrulline, 3-guanidinopropionic acid, 4-{[amino(imino)methyl]amino}butanoic acid, 6-{[amino(imino)methyl]amino}hexanoic acid, 2-Amino-3-guanidinopropionic acid, Arginine hydroxamate, Agmatine (CAS #: 2482-00-0), and NG-Methyl-arginine, preferably, the basic amino acids are, independently of each other, selected from the group consisting of lysine (K), histidine (H) and arginine (R).

9. The compound of claim 1, wherein X¹, X², Y¹, Y², Z¹ and Z², are independently of each other, at physiological pH, positively or negatively charged amino acids, and wherein the negatively charged amino acids are, independently of each other, selected from the group consisting of homoglutamic acid, a sulfonic acid derivative of Cys, cysteic acid, homocysteic acid, aspartic acid (D), glutamic acid (E), preferably, the acidic amino acids are, independently of each other, selected from aspartic acid and glutamic acid.

10. Complex comprising
(a) a radionuclide, and
(b) the compound of claim 1 or a pharmaceutically acceptable salt or solvate thereof.

11. The complex of claim 10, wherein, the radionuclide is selected from the group consisting ⁸⁸⁹Zr, ⁴⁴Sc, ¹¹¹In, ⁹⁰Y, ⁶⁶Ga, ⁶⁷Ga, ⁶⁸Ga, ¹⁷⁷Lu, ^{99m}Tc, ⁶⁰Cu, ⁶¹Cu, ⁶²Cu, ⁶⁴Cu, ⁶⁶Cu, ⁶⁷Cu, ¹⁴⁹Tb, ¹⁵²Tb, ¹⁵⁵Tb, ¹⁵³Sm, ¹⁶¹Tb, ¹⁵³Gd, ¹⁵⁵Gd, ¹⁵⁷Gd, ²¹³Bi, ²²⁵Ac, ²³⁰U, ²²³Ra, ¹⁶⁵Er, ⁵²Fe, ⁵⁹Fe, and radionuclides of Pb (such as ²⁰³Pb and ²¹²Pb, ²¹¹Pb, ²¹³Pb, ²¹⁴Pb, ²⁰⁹Pb, ¹⁹⁸Pb, ¹⁹⁷Pb).

12. A pharmaceutical composition comprising a compound of claim 1.

13. A method for treating or preventing PSMA-expressing cancer and/or metastases thereof, in particular prostate cancer and/or metastases thereof with a compound of claim 1.

14. A method for diagnosing PSMA-expressing cancer and/or metastases thereof, in particular prostate cancer and/or metastases thereof with a compound of claim 1.

15. (canceled)
